(19) United States
(12) Patent Application Publication
(10) Pub. No.: US 2006/0252036 A1
(43) Pub. Date: Nov. 9, 2006

(54) Alteration of rab38 function to modulate mammalian pigmentation

(75) Inventors: William J. Pavan, Derwood, MD (US); Stacie K. Loftus, Great Falls, VA (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: PAVAN AND LOFTUS

(21) Appl. No.: 10/501,611

(22) PCT Filed: Jan. 17, 2003
(86) PCT No.: PCT/US03/01622

§ 371 Date: Oct. 20, 2005

(30) Foreign Application Priority Data
Jan. 18, 2002 (US) 60349929

(51) Int. Cl. C12Q 1/68

(52) U.S. Cl. 435/6

(57) ABSTRACT

The present invention relates to compositions and methods involving melanocytes. In particular, the present invention provides compositions and methods involving RAB38 and mutant RAB38, suitable for use in modulation of pigmentation and for use in determining the means to diagnose and/or treat conditions associated with disorders in pigmentation.

### CROSS-REFERENCES TO RELATED APPLICATIONS

This application claims priority from U.S. Provisional Patent Application No. 60/349,929 filed Jan. 18, 2002, and incorporates the contents thereof.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### FIELD OF THE INVENTION

The present invention relates to compositions and methods involving melanocytes. In particular, the present invention provides compositions and methods involving RAB38 and mutant RAB38, suitable for use in modulation of pigmentation and for use in determining the means to diagnose and/or treat conditions associated with disorders in pigmentation.

### BACKGROUND OF THE INVENTION

Melanocytes are specialized pigment producing cells that are responsible for coloration of skin, eyes and hair. Coat color alterations resulting from melanocyte defects are easily identifiable in mice. These mouse mutants are proving valuable for the identification of candidate human disease genes and for the elucidation of mechanisms underlying cellular function. To date, there are approximately 100 loci in the mouse that, when mutated, affect pigmentation. However, the underlying genetic defect has not been identified in about 60 of these loci (See, The Jackson Laboratory's Mouse Genome Informatics web site).

Disorders with reduced pigmentation can be placed into two groups according to whether they affect melanocyte differentiation or whether they affect the function of the pigment producing organelle in the melanocyte, the melanosome. Examples of the first group include Piebaldism and Waardenburg Syndrome, characterized by a localized absence of melanocytes resulting in “white patch” patterns. Genes affected in these disorders, KIT, MITF, PAX3, SOX10, EDNRB, EDN3, are involved in specification, migration and survival of the melanocyte lineage (Jackson, Hum Mol Genet 6:1613 1624 [1997]). Mouse models of these disorders have characteristic spotted coat patterns (Jackson, Hum Mol Genet 6:1613 1624 [1997]). Oculocutaneous albinism (OCA) I-IV, Chediak-Higashi Syndrome (CHS), Hermansky-Pudlak Syndrome (HPS) I-III and Griscelli syndrome (GS) correspond to the second group. The molecular defects contributing to the reduced pigmentation in OCA occur in genes (TYR, TYRPI, P and AIM1]) that mainly effect melanosome formation and the amount and type of melanin pigment formed (King et al., in Scriver et al, (eds.) The metabolic basis of inherited disease, 7th ed. (McGraw Hill, New York) gyp. 4353-4392 [1995]); and Newton et al., Am J Hum Genet 69:981-988 [2001]). Genes responsible for HPS, CHS and GS are involved in the regulation of vesicle traffic including melanosomes within the cell and include HPSl, AP3, HPS3, CHSI; MYO5A and RAB27A (Jackson, Hum Mol Genet 6:1613-1624 [1997]; and Marks and Seabra, Nat Rev Mol Cell Biol 2:738-748 [2001]).

Although many genes have been associated with pigmentation disorders, in view of the genetic heterogeneity of these disorders in both mice and humans, there is a need in the art to identify additional candidate disease genes in these and other species.

### BRIEF SUMMARY OF THE INVENTION

The present invention relates to compositions and methods involving melanocytes. In particular, the present invention provides compositions and methods involving RAB38 and mutant RAB38, suitable for use in modulation of pigmentation and for use in determining the means to diagnose and/or treat conditions associated with disorders in pigmentation.

The present invention provides an isolated nucleic acid that comprises a sequence selected from the group consisting of SEQ ID NO:9, SEQ ID NO:10, SEQ ID NO: 11, and SEQ ID NO:12. In some embodiments, the nucleic acid is deoxyribonucleic acid. In other embodiments, the nucleic acid is the complement of SEQ ID NO:9, SEQ ID NO:10, SEQ ID NO:11, and SEQ ID NO:12. In related embodiments, a vector comprising the nucleic acid is provided. Also provided is a host cell comprising the vector. Additionally, the present invention provides a protein encoded by the nucleic acid sequence set forth in SEQ ID NO:9.

In some embodiments, the present invention provides methods for detecting mutations in Rab38 comprising the steps of: amplifying at least a portion of Rab38 from genomic DNA to yield a Rab38 amplification product; purifying the Rab38 amplification product; and sequencing the Rab38 amplification product. In preferred embodiments, the amplifying is accomplished using a polymerase chain reaction. In related embodiments, the at least a portion of Rab38 genomic DNA is selected from the group consisting of at least one Rab38 exon, at least one Rab38 intron, the Rab38 5′ untranslated sequence, and the Rab38 3′ untranslated sequence. In some particularly preferred embodiments, the at least one Rab38 exon is selected from the group consisting of Rab38 exon 1, Rab38 exon 2, and Rab38 exon 3. In some embodiments, the genomic DNA is mammalian genomic DNA. Also provided are embodiments where the purifying is accomplished via size selection.

The present invention further provides methods for detecting mutations in Rab38 comprising the steps of amplifying at least a portion of Rab38 from genomic DNA to yield a Rab38 amplification product; digesting the Rab38 amplification product to yield a digested Rab38 amplification product; and electrophoresing the digested Rab38 amplification product. In preferred embodiments, the amplifying is accomplished using a polymerase chain reaction. In some embodiments, the at least a portion of Rab38 genomic DNA is selected from the group consisting of at least one Rab38 exon, at least one Rab38 intron, the Rab38 5′ untranslated sequence, and the Rab38 3′ untranslated sequence. In preferred embodiments, the at least one Rab38 exon is selected from the group consisting of Rab38 exon 1, Rab38 exon 2, and Rab38 exon 3. In particularly preferred embodiments, the genomic DNA is mammalian genomic DNA.

The present invention also provides methods for screening for biologically active agents to modulate RAB38 activity, comprising the steps of providing: melanocytes comprising RAB38 activity, and a candidate agent; and exposing the melanocytes to the candidate agent to yield treated melanocytes; and measuring the modulation of the RAB38 activity of the treated melanocytes by the candidate agent. In some embodiments, the RAB38 activity comprises GTPase activity. In some related embodiments, the RAB38 activity comprises GTP binding activity or GDP release. In some preferred embodiments, the RAB38 activity comprises TYRP1 trafficking to melanosomes or RAB38 trafficking to melanosomes.

In some embodiments, the present invention provides kits for screening for biologically active agents that modulate RAB38 activity, comprising: plurality of melanocytes comprising RAB38 activity, wherein the melanocytes are provided within a container, and instructions for determination of RAB38 activity in the melanocytes. In some preferred embodiments, kits further comprise the means to analyze RAB38 activity. In some related embodiments, the means to analyze RAB38 activity comprises an assay to assess GTPase activity, an assay to assess GTP binding activity, an assay to assess GDP release, an assay to assess TYRP 1 trafficking to melanosomes, or an assay to assess RAB38 trafficking to melanosomes.

The present invention also provides kits for the detection of mutations in RAB38 comprising at least two primer sequences suitable for amplification of at least a portion of RAB38, and instructions for utilizing the kit. In some preferred embodiments, the primer sequences are selected from the group consisting of SEQ ID NO:17, SEQ ID NO:18, SEQ ID NO:19, SEQ ID NO:20, SEQ ID NO:21, and SEQ ID NO:22. In some related embodiments, the kit is suitable for use in the polymerase chain reaction. The invention also provides embodiments further comprising reagents for digesting nucleic acid.

In some embodiments, the present invention also provides kits for diagnosing defects in melanosome function, comprising melanocytes comprising RAB38 and instructions for assessing defects in melanosome function. In some preferred embodiments, the kits further comprise the means to analyze RAB38 activity. In some related embodiments, the means to analyze RAB38 activity comprises an assay to assess GTPase activity, an assay to assess GTP binding activity, an assay to assess GDP release, an assay to assess TYRP 1 trafficking to melanosomes or an assay to assess RAB38 trafficking to melanosomes.

### BRIEF DESCRIPTION OF THE DRAWINGS

FIG. 1 depicts a microarray expression profile of various pigmentation control genes and Rab38. Cluster analysis identified nine genes known to be involved in pigmentation. Columns to the right list mouse and human pigmentation disorders corresponding to a given gene. Rab38 (arrow) was found within the same expression profile cluster as the nine control genes known to be involved in pigmentation. Hybridized sample cell lines are listed across the top of the expression profile. Relative gene expression is evaluated as a calibrated ratio (sample cell line/MnSOD6c11 reference). Pseudocolor scale for ratio values is shown below.

FIG. 2 illustrates that RAB38, like known melanogenic enzymes, is expressed in the retinal pigmented epithelium (RPE). All panels show the eye at embryonic day E11.5. The melanogenic enzymes expressed in the RPE at this developmental stage include tyrosinase (Tyr; Panel A), tyrosinase related protein 1 (Tyrpl; Panel B), and dopachrome tautamerase (DCT/TTyrp2; Panel C). Additional control genes from the microarray cluster data that are expressed in the RPE include melastatnl (Mlsn; Panel D) and Aiml/Matp (Panel E). Rab38 is expressed in the RPE at E11.5 (Panel F) and at E10.5 and E12.5. The scale bar equals 100 pm.

FIG. 3 illustrates the Rab38 map location and the phenotype of the chocolate (cht/cht) mouse. Panel A provides a map of human chromosome 11 showing that human Rab38 maps 1.4 Mb distal to tyrosinase and 2.5 cM proximal to EED genes. In the corresponding mouse chromosome 7, syntenic region gene order is conserved (e.g., the cht locus maps to the same interval as Rab38). Panel B provides a photograph of C57B1/6J+/+ (left side, black) and C57B1/6J Rab38^{cht}/Rab38^{cht} (right side, brown) mice. Panel C shows the eyes from 2 day old wildtype C57B16/J+/+ mice with normal pigmentation, while Panel D shows that the eyes from Rab38^{cht}/Rab38^{cht} mice exhibit much less pigmentation.

FIG. 4 indicates that the Rab38 mutation causes the chocolate (cht) mouse phenotype. Panel A provides a comparison of Rab38 sequence between wildtype C57B16/J+/+ DNA (SEQ ID NO: 1) and mutant C57BI/6J Rab38^{cht}/+DNA (SEQ ID NO:2), revealing a G146T nucleotide change (arrow) in the cht allele. This nucleotide change was never seen in eight additional inbred strains analyzed. Panel B illustrates that the G146T mutation creates a SexA1 restriction enzyme site in C57B1/6J Rab38^{cht}/Rab38^{cht} DNA and ablates a BsaJI restriction site present in wildtype Rab38 sequence. A 216 by region surrounding the G146T nucleotide mutation was amplified from both C57B1/6J+/+ DNA and C57B16/J Rab38^{cht}/Rab38^{cht} DNA. SexA1 digests the PCR fragment of C57B1/6J Rab38^{cht}/Rab38^{cht} (lane 1), but not C57B1/6J+/+ (lane 2); BsaJ1 digests the PCR fragment of C57B16/J+/+ (lane 4), but not C57B1/6J Rab38^{cht}/Rab38^{cht} (lane 3).

FIG. 5 shows that RAB38 G19 is located in the GTP binding pocket. Panel A provides the 3 dimensional location of the amino acid G19 of RAB38 in relation to the nucleotide binding site, as determined using the molecular modeling database (MMDB; Wang et al., Nucleic Acids Res 28:243-245 [2000]), based upon the crystal structure for RAB3a (MMDB 10125; and Dumas et al., Structure Fold Des 7:413-423 [1999]). Overlaying the RAB38 sequence with that of RAB3a identified amino acid S32 of RAB3a as being equivalent to G19 of RAB38. The program Cn3D 3.0 was used to indicate the location of the RAB38 G19 (white), predicting interaction with the bound nucleotide. Protein structure is indicated by color: green, \acute{α}-helices; gold, β sheet; blue, random coils; white, site of RAB3a S32 equivalent to RAB38 G19 located at the nucleotide binding site; grey, Mg⁺⁺ ion; red grey, GppNHp nucleotide analog. Panel B provides alignments of the highly conserved N-terminal region including human RAB38 (NP_{—}071732; and SEQ ID NO:3), rat RAB38 (AAA42000; and SEQ ID NO:4), and mouse RAB38 (AK009296.1; and SEQ ID NO:5) amino acid sequences; and human RAB3a (P20336; and SEQ ED NO:6), human RABS (F34323; and SEQ ID NO:7) and human N RAS (TVHURA; and SEQ ID NO:8) amino acid sequences. Sequence alignment was done using the ClustalW algorithm (Smith et al., Genome Res 6:454-462 [1996]). Bars indicate highly conserved regions that occupy the nucleotide binding pocket, observed in the X ray crystal structure of RAB3a (Ostermeier and Brunger, Cell 96:363-374 [1999]). Black denotes sequence identity, grey denotes sequence conservation, and red denotes the conserved amino acid that is mutated in the chocolate mice.

FIG. 6 illustrates that cht/cht melanosomes are similar in morphology to Tyrpl^{b} melanosomes. Bright field images of melanosomes are shown from the periphery of primary cultured melanocytes, isolated from C57B16/J+/+ mice in Panel A, and from C57B1/6J Rab38^{cht}/Rab38^{cht} mice in Panel B. Melanosomes from wildtype melanocytes are oval and darkly pigmented, while those from C57B1/6J Rab38^{cht}/Rab38^{cht} melanocytes are smaller, more circular and less pigmented, resembling melanosomes from Tyrpl^{b}/Tyrpl^{b}, melan b cells. The scale bar equals 2 μm.

FIG. 7 shows that RAB38 is a melanosomal protein needed for appropriate TYRP1 trafficking. Bright field and matching confocal images of identical exposure of melanosomes in the periphery of primary melanocytes cultures are provided in the upper and lower panels respectively. Panels A and B provide images of C57B1/6J+/+ melanosomes, Panels C and D provide images of C57BI/6J Rab38^{cht}/Rab38^{cht} melanosomes, and Panels E and F provide images of melanosomes from melan-a cells transfected with a GFP RAB38 expression construct. TYRP1 distribution was revealed by MEL5 staining in Panels B and D, while GFP RAB38 immunofluorescence shown in Panel F, demonstrates co localization of the GFP RAB38 signal with the highly pigmented, end stage melanosomes. The scale bar for Panels A and D equals 1.6 μM, while that for Panels E and F equals 2.4 μM.

### DETAILED DESCRIPTION OF THE INVENTION

The present invention relates to compositions and methods involving melanocytes. In particular, the present invention provides compositions and methods involving RAB38 and mutant RAB38, suitable for use in modulation of pigmentation. In addiotion the method can be used to diagnose and/or treat conditions associated with disorders in pigmentation.

In order to meet a need in the art for identification of additional pigmentation disease genes, expression profile analysis using cDNA microarrays was conducted done to develop the present invention. Expression profile analysis was utilized for the development of the present invention, as it is a powerful tool for organizing the common patterns found among thousands of gene expression measurements, and for identifying genes with similar distinctive expression patterns among multiple experimental samples (Eisen et al., Proc Natl Acad Sci USA 95:14863-14868 [1998]). Analysis of genes contained within a cluster has revealed that these genes are often functionally related within the cell (Eisen and Brown, Methods Enzymol 303:179-205 [1999]; and Mody et al., Proc Natl Acad Sci USA 98:8862-8867 [2001]). As detailed below, by using this approach Rab38 was identified as a candidate pigmentation gene. Further analysis confirmed that RAB38 is a melanosomal protein, mutated in the mouse pigmentation mutant, chocolate (cht), and important for the sorting of the melanosomal protein TYRP 1 in melanocytes. These experiments conducted during the development of the present invention provide the first successful use of microarray expression profiling to identify a mammalian pigmentation disease gene.

Expression Profile Analysis Identifies RAB38 as a chocolate Candidate Gene

In order to identify novel and uncharacterized genes involved in melanocyte function and disease, a collection of cDNA clones to be used for expression profile and functional analyses were generated (Loftus et al., Proc Natl Acad Sci USA 96:9277-9280 [1999]). cDNA clones from IMAGE consortium library 2NbHM (See, The National Center for Biotechnology Information's web site for UniGene Library No. 198) were previously shown to be appropriate for gene expression studies aimed at understanding melanocyte development and function (Loftus et al, Proc Natl Acad Sci USA 96:9277-9280 [1999]; and Loftus and Pavan, Pigment Cell Res 13:141-146 [2000]). For this analysis, 4356 cDNA clones from library 2NbHM were printed to glass slides. A total of 17 cell lines representing neural crest and non-neural crest derived tissues were used in this analysis. Included were eleven melanoma cell lines (Bittner et al., Nature 406:536-540 [2000]), three rhabdomyosarcoma cell lines, one glioblastoma cell line, HeLa cells, and 293T cells. Array hybridizations for each of these cell lines were performed in a pair wise fashion, using RNA from cell line MnSOD6 cl1 as a reference. MnSOD6 cl1 is an amelanotic melanoma cell line rendered non-tumorigenic by the introduction of a region of human chromosome 6 (Trent et al, Science 247:568-571 [1990]). MnSOD6 cl1 has been used previously for expression profile analysis of melanoma lines using a different set of cDNA clones (Bittner et al., Nature 406:536-540 [2000]).

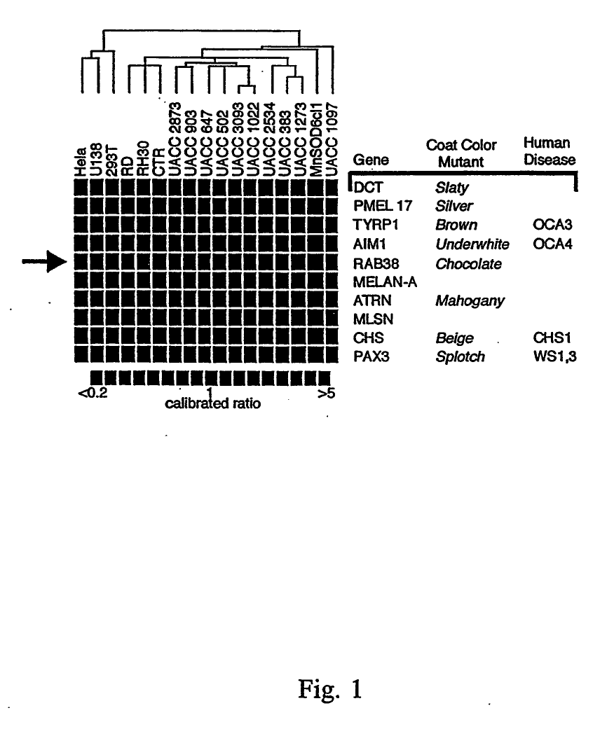

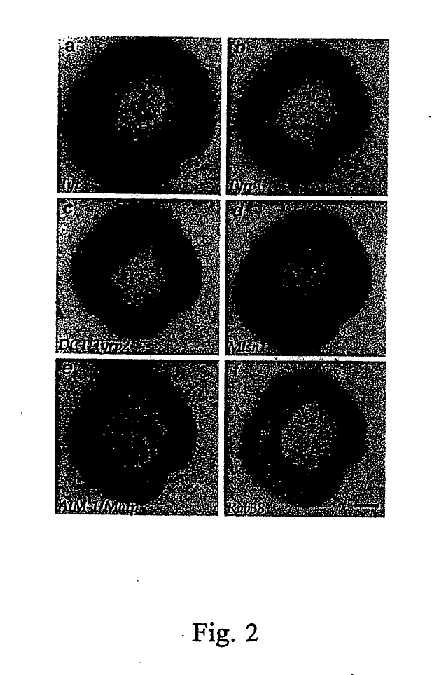

Hierarchical cluster analysis found nine genes (DCT, TYRP1, PMEL17, AIM-1, MELAN A/MARTI, MLSN, ATRN, PAX3 and CHSl), previously known to be involved in melanocyte function, to cluster together (See, FIG. 1). On closer analysis of the hierarchical clustering data an additional gene, Rab38, was found to have a similar expression variation to these melanocyte genes (See, FIG. 1). Four of the nine melanocyte genes examined (TYRP1, DCT, MLSN and AIM1]) were expressed in the melanocytes of the retinal pigment epithelium (RPE) at E11.5 (See, FIG. 2). Consistent with the placement of Rab38 within this cluster of genes, whole mount in situ analysis demonstrated that Rab38 was also expressed in the melanocytes of the RPE at this age (See, FIG. 2). Northern blot analyses revealed that Rab38 expression was restricted to the melanocyte derived cell lines (Jager et al., Cancer Res 60:3584-3591 [2000]).

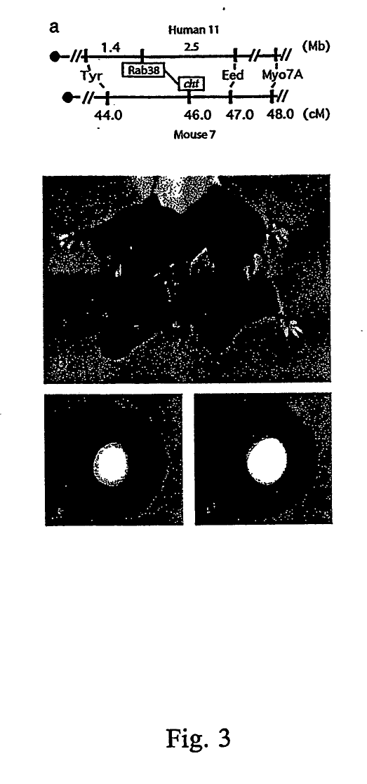

Utilizing the recently available human genome sequence, Rab38 was determined to be located on human chromosome 11, flanked proximally by TYR and distally by EED and MY07A (See, FIG. 3, Panel A). A conserved linkage group on mouse chromosome 7 was identified by comparison of the human genome map with the mouse genome mapping data (See, The Jackson Laboratory's web site for Mammalian Homology Query). Closer analysis of loci in the mouse conserved linkage group indicated that an uncloned mouse pigmentation mutant, cht, was contained within this interval (See, FIG. 3, Panel A; and Potter and Rinchik, Mamm Genome 4:46-48 [1993]). The cht mutation arose as a spontaneous, isogenic mutation on an inbred C57B1/6J background and has been maintained on this background since 1984 (Macpike and Mobraaten, Mouse News Lett 700:86 [1984]). Cht/cht mice are identifiable at birth by lighter skin and eyes, and at weaning by a deep brown coat color when compared to the C57B1/6J parental strain (See, FIG. 3, Panels B and C). Thus, Rab38 was implicated as a candidate gene for the cht locus based on genomic map positions and the expression of Rab38 in cell types affected in cht/cht mice, RPE and melanocyte derivatives.

Mutation of Rab38 Causes Melanocyte Defects in cht/cht Mice

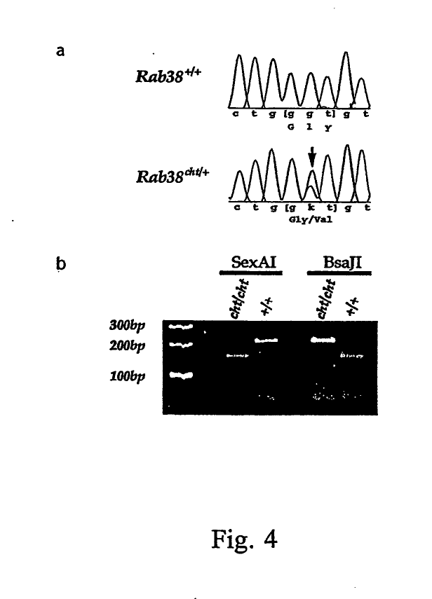

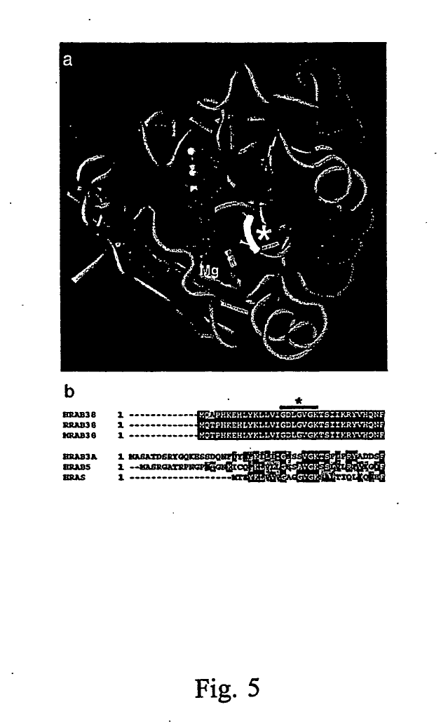

Genomic sequence flanking exon/intron boundaries for the three mouse Rab38 exons was obtained from mouse trace archive genomic sequence (See, National Center for Biotechnology Information web site for Trace Archive Querying). DNA from C57B1/6J chtl+ animals was obtained from Jackson Laboratories Mouse DNA Resource, amplified using genomic Rab38 primers and directly sequenced. In exon 1, a G146T nucleotide mutation was identified in the cht allele (See, FIG. 4, Panel A). This sequence alteration was confirmed by restriction digest in multiple cht/cht DNA samples, as the resulting nucleotide substitution changes a BsaJI site (CCNNGG) to a SexA1 restriction site (ACCWGGT) (See, FIG. 4, Panel B). This sequence alteration was not detected in analysis of 8 additional inbred mouse strains (CAST/Ei, SPRET/Ei, 129/SVJ, FVB/NJ, AKR/J, A/J, DBA/1J, BALB/cJ. The RAB38 protein demonstrates highly conserved amino acid identity between phyla: human/rat (96.2%), human/mouse (93.8%), rat/mouse (95.2%) (See, FIG. 4, Panel B). The G19V cht mutation is located within the highly conserved phosphate/Mg²⁺ (PM) domain and is predicted to directly contact GTP in the nucleotide binding pocket (See, FIG. 5, Panels A and B).

Rab38^{cht} Results in a Decrease in the Efficiency of Targeting Tyrp1 to End Stage Melanosomes

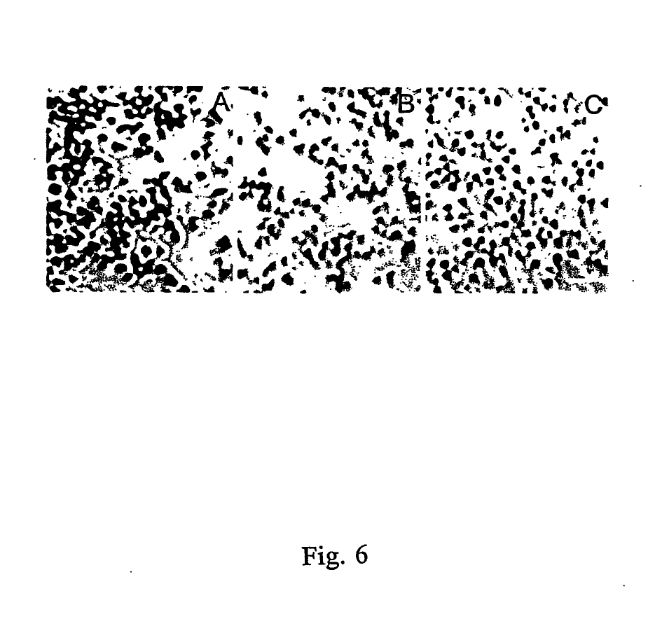

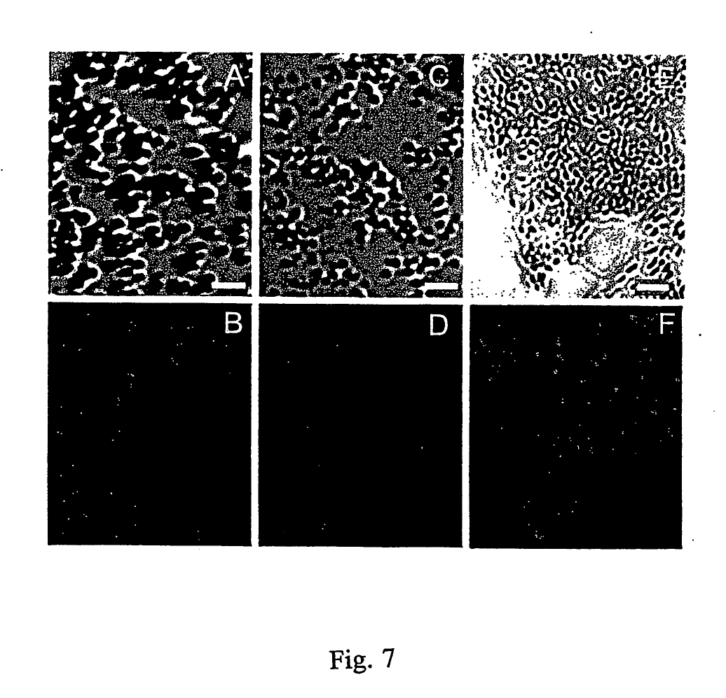

Analysis of melanocytes cultured from newborn mice revealed that C57B1/6J Rab38^{cht}/Rab38^{cht} melanocytes contain, small, circular melanosomes with a brown hue similar to those observed in C57B1/6J Tyrpl^{b}/Tyrpl^{b} melanocytes (See, FIG. 6, Panel C; and Hearing et al., J Ultrastruct Res 43:88-106 [1973]), but distinct from the intensely black, oval melanosomes seen in C57B1/6J+/+melanocyte cultures (See, FIG. 6, Panels A and B). Given that Tyrpl mutations cause the switch from black to brown pigment in brown mice, and given that Rab GTPases play a central role in protein trafficking (Schimmoller et al., J Biol Chem 273:22161-22164 [1998]; and Chavrier and Goud, Curr Opin Cell Biol 11:466-475 [1999]), the targeting of TYRPI to the melanosome was contemplated to be defective in Rab38^{cht}/Rab38^{cht} melanocytes. Consistent with this, endstage melanosomes in the Rab38^{cht}/Rab38^{cht} melanocytes stain much more weakly for TYRP1 than do end stage melanosomes in the control melanocytes (See, FIG. 7, Panels A D). Additionally, GFP tagged RAB38 co-localizes with end stage melanosomes in wild type cells (See, FIG. 7, Panels E and F). Thus, RAB38 is contemplated to regulate traffic of vesicular intermediates that move Tyrp1 from the trans-golgi network (TGN) to end stage melanosomes.

Rab38^{cht}/Rab38^{cht} Does Not Result in Platelet Storage Defects

Subsets of mouse coat color mutants with mutations in genes involved in vesicular trafficking, such as pale ear and beige, also cause platelet aggregation defects modeling HPS and CHS respectively. To further analyze the pathology of Rab38^{cht}/Rab38^{cht} mice, bleeding times were measured as an assay of platelet function. No difference was observed between wildtype and Rab38^{cht}/Rab38^{cht} mice (2.53 vs. 2.41 minutes; p=0.7). This observation is consistent with Rab38^{cht}/Rab38^{cht} being a genocopy for OCAIII (Tyrp1^{b}) mouse model, but not for either HPS or CHS. Taken together, the data presented herein indicate that RAB38 is required for the efficient targeting of TYRP1 to pigmented melanosomes, and suggests that Rab38^{cht}/Rab38^{cht} is a genocopy of the Tyrp1^{b}/Tyrp1^{b} OCAIII mouse model.

Rab38 is a Melanocyte Pigmentation Gene in Mammals

Thus, by using cDNA microarray expression profiling, Rab38 has been identified as an important gene involved in melanocyte pigmentation. Hierarchical clustering of expression patterns grouped Rab38 with nine previously identified melanocyte genes known to function in a melanocyte specific fashion. These genes include DCT, TRYP1, and PMELl7 which are essential for melanosome function; MELAN-A/MART1 and MLSN which are important melanoma antigens (Chen et al., Proc Natl Acad Sci USA 93:5915-5919 [1996]; and Duncan et al., J Clin Oncol 19:568-576 [2001]); AIM-1 which has recently been identified as the gene responsible for B in medaka (Fukamachi et al., Nat Genet 28:381-385 [2001]), underwhite in mice (Newton et al, Am J Hum Gent 69:981-988 [2001]), and OCA4 in humans (Newton et al., Am J Hum Gent 69:981-988 [2001]); CHS1 which functions in melanosome/lysosome vesicle trafficking (Introne et al., Mol Genet Metab 68:283-303 [1999]); and PAX3, a paired box transcription factor that regulates melanocyte gene expression (Watanabe et al., Nat Genet 18:283-286 [1998]; Potterf et al., Human Genet 107:1-6 [2000]; and Hornyak et al., Mech Dev 101:47-59 [2001]), including expression of TYRPl (Galibert et al., J Biol Chem 274:26894-26900 [1999]). Mutations in seven of these genes have been identified in human and/or marine disorders associated with variations in pigmentation (See, FIG. 1).

Rab38 was assessed as a candidate gene for the cht locus for three reasons. First, comparative genomic analysis predicted a co localization of the human Rab38 gene to the region of the cht locus in the mouse genome. Second, the expression of Rab38 was found to be restricted to those cell types affected in cht/cht mice (See, FIG. 2, and Jager et al., Cancer Res 60:3584-3591 [2000]). Finally, Rab38 is a member of a family of proteins that are known to play a crucial role in vesicular trafficking (Nielsen et al., Nat Cell Biol 1:376-382 [1999]; and Scott and Zhao, J Invest Dermatol 116:296-304 [2001]).

Sequence analysis of the Rab38 coding region from cht mice revealed a G146T transversion in exon 1. This sequence alteration is likely to be the causative mutation, since this allele arose as a spontaneous mutation on a C57B1/6J background. Moreover, the G146T alteration results in a Gly to Val substitution within the GTP binding pocket of RAB38. Crystal structure analysis of RAB3A, which is used as a model for other Rab proteins, predicts that this amino acid residue directly contacts the GTP within the nucleotide binding pocket (Dumas et al., Structure Fold Des 7:413-423 [1999]). Furthermore, a mutation of the analogous amino acid residue in RAB5, a Rab that regulates the homotypic fusion of endosomes, results in an increased rate of GDP dissociation in vitro, and the stimulation of endosome fusion in vivo (Li and Liang, Biochem J 355:681-689 [2001]). Additional support for the functional relevance of this mutation comes from studies of the Ras protein. Substitutions in Ras at the analogous G13 residue, including the same G to V mutation as in Rab38^{cht}, have been identified in acute myeloid leukemia (Bos et al., Nature 315:726-730 [1985]; and Stirewalt et al., Blood 97:3589-3595 [2001]). Based upon the analyses disclosed herein and on these observations, the Gly to Val mutation in RAB38 is contemplated to disrupt RAB38 function in vivo.

The coat color of Rab38^{cht}/Rab3^{cht} mice closely resembles that of the brown (Tyrp1^{b}/Tyrp1^{b}), OCAII mouse model. The brown mouse model contains a defect in a melanin biosynthesis gene Tyrp1, resulting in a coat color change of the C57BL/6J mouse from black to brown. TYRP is a melanosomal membrane glycoprotein, which functions both as a DHICA oxidase enzyme and to provide structural stability to TYR in the melanogenic enzyme complex. TRYPI is believed to transit from the trans-golgi network (TGN) to stage II melanosomes via clatherin coated vesicles, possibly by first passing through an uncharacterized sorting compartment (Marks and Seabra, Nat Rev Mol Cell Biol 2:738-748 [2001]). Based upon the similar coat phenotype and predicted Rab protein function, RAB38 is contemplated to be specifically involved in trafficking of melanosomal proteins like TYRP1, to the melanosome. Consistent with this, GFP tagged RAB38 co localizes with melanosomes in pigmented melanocyte lines in culture and TYRP 1 is inefficiently targeted to pigmented end stage melanosomes in Rab38^{cht}/Rab38^{cht} melanocytes. The brown coat color observed in Rab38^{cht}/Rab38^{cht} mice is contemplated to be the result of a reduced amount of melanosomal TYRP1. Thus, RAB38 is implicated in the vesicle trafficking required for proper targeting of proteins, such as TYRP1, to melanosomes.

The formation of melanosomes and melanin pigment deposition within them requires a series of specific vesicular trafficking steps (King et al, in Scriver et al. (eds.) The Metabolic Basis of Inherited Disease, 7th ed. (McGraw Hill, N.Y.) pp. 4353-4392 [1995]; and Marks and Seabra, Nat Rev Mol Cell Biol 2:738 748 [2001]). Comparison of the phenotype of Rab38^{cht} mice to other mouse mutants where defects in the trafficking of proteins has been identified is contemplated to provide insight into the site of action of RAB38. Four genes involved in HPS (HPS1, AP3 and HPS3 and HPS4), when mutated result in the mouse models pale ear (ep), mocha, cocoa, and light ear, respectively. For each of these mouse models the color of melanin produced by the melanosome is lighter in color or of a brown hue. Interestingly, similar to what is seen in Rab38^{cht}/Rab38^{cht} derived melanocytes, melanocytes from Hps1^{ep}/Hps1^{ep} mutants also exhibit a mislocalization of TYRP 1 into membraneous complexes rather than pre melanosomes (Sarangarajan et al., J Invest Dermatol 117:641-646 [2001]), again yielding a brown mouse. In addition to melanosome pigment defects, HPS mice also exhibit enlargement of melanosomes and lysosomes, and reduced platelet cell aggregation (Swank et al., Pigment Cell Res 13:59-67 [2000]; and Introne et al., Mol Genet Metab 68:282-303 [1999]). This suggests the involvement of HPS genes in early vesicle sorting events that affect lysosomes, as well as melanosomes. However, it appears that Rab38^{cht} is not in the same class of mutants as those of the HPS mouse models, since Rab38^{cht}/Rab38^{cht} mice do not exhibit enlarged melanosomes or lysosomes, or defects in platelet function. Thus, although both HPS1 and RAB38 appear to be involved in the proper sorting of TYRP1, this regulation appears to be occurring at different steps in the trafficking process. Since RAB38 appears to affect melanosome trafficking only, RAB38 is contemplated to be involved in vesicle trafficking downstream of the HPS genes.

Rab38^{cht} mice appear to be a genocopy of the TYRP1^{b}, OCA mouse model, due to the essential role of RAB38 in proper TYRP1 trafficking to late stage melanosomes, thus mimicking the cellular and clinical phenotype. OCA is a heterogeneous genetic disorder that has been associated with mutations in TYR (OCAI), P (OCAII) TYRP1 (OCAIII) and AIM1 (OCAIV). However, approximately 10% of patients clinically diagnosed with OCA do not have mutations in any of these genes. Given the heterogeneity of OCA and the predicted role of RAB38 in TYRPI sorting, Rab38 is contemplated to be a candidate gene for patients with OCA, particularly when a molecular defect in TYR, P, TYRP1 or AIM1 has not been found.

High Throughput Screening for Modulators of RAB38 Activity

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### a. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i. e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 12331250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random biooligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad Sci. USA 90: 69096913), vinylogous polypeptides (Hagihara et al. (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem Soc. 116: 2661), oligocarbamates (Cho, et al., (1993) Science 261:1303), and/or peptidyl phosphonates (Campbell et al, (1994) J. Org. Chem 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

### b. High Throughput Assays of Chemical Libraries

Any of the assays for compounds inhibiting the virulence described herein are amenable to high throughput screening. As described above, having identified the nucleic acid associated with virulence, likely drug candidates either inhibit expression of the gene product, or inhibit the activity of the expressed protein. Preferred assays thus detect inhibition of transcription (i.e., inhibition of RAB38 mRNA production) by the test compound(s), inhibition of protein expression by the test compound(s), or binding to the gene (e.g., gDNA, or cDNA) or gene product (e.g., RAB38 mRNA or expressed protein) by the test compound(s). Alternatively, the assay can detect inhibition of the characteristic activity of the gene product or inhibition of or binding to a receptor or other transduction molecule that interacts with the gene product

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

Methods of Inhibiting RAB38 Activity

Methods of the invention include methods of inhibiting RAB38 activity. Any method known in the art may be used, all that matters is that RAB38 activity be reduced or eliminated compared to a wild type state or a control. Inhibitors of RAB38 activity may act at any step of the expression process.

RAB38 activity may be inhibited by a number of methods including the use of ribozymes, antisense molecules, siRNA, or small chemical blockers of RAB38 activity. Antisense and RNAi Polynucleotides

In certain embodiments, the activity of RAB38 protein is down-regulated, or entirely inhibited, by the use of an inhibitory or antisense polynucleotide, e.g., a nucleic acid complementary to, and which can preferably hybridize specifically to, a coding mRNA nucleic acid sequence, e.g., a RAB38 mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species. Analogs are comprehended by this invention so long as they function effectively to hybridize with the cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences. A preferred antisense molecule is for a RAB38 molecules or for a ligand or activator thereof. Antisense or sense oligonucleotides, according to the present invention, comprise a fragment generally at least about 14 nucleotides, preferably from about 14-30 nucleotides. The ability to derive an antisense or a sense oligonucleotide, based upon a cDNA sequence encoding a given protein is described in, e.g., Stein and Cohen (1988) Cancer Res. 48:2659-2668; and van der Krol, et al. (1988) BioTechniques 6:958-976.

RNA interference is a mechanism to suppress gene expression in a sequence specific manner. See, e.g., Brumelkamp, et al. (2002) Sciencexpress (21Mar. 2002); Sharp (1999) Genes Dev. 13:139-141; and Cathew (2001) Curr. Op. Cell Biol. 13:244-248. In mammalian cells, short, e.g., 21 nt, double stranded small interfering RNAs (siRNA) have been shown to be effective at inducing an RNAi response. See, e.g., Elbashir, et al. (2001) Nature 411:494-498. The mechanism may be used to downregulate expression levels of identified genes, e.g., treatment of or validation of relevance to disease.

In addition to antisense polynucleotides, ribozymes can be used to target and inhibit transcription of RAB38 nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto, et al. (1994) Adv. in Pharmacology 25: 289-317 for a general review of the properties of different ribozymes).

The general features of hairpin ribozymes are described, e.g., in Hampel, et al. (1990) Nucl. Acids Res. 18:299-304; European Patent Publication No. 0 360 257; U.S. Pat. No. 5,254,678. Methods of preparation are described in, e.g., WO 94/26877; Ojwang, et al. (1993) Proc. Natl. Acad. Sci. USA 90:6340-6344; Yamada, et al. (1994) Human Gene Therapy 1:39-45; Leavitt, et al.(1995) Proc. Natl. Acad. Sci. USA 92:699-703; Leavitt, et al. (1994) Human Gene Therapy 5:1151-120; and Yamada, et al. (1994) Virolog 205: 121-126.

Polynucleotide modulators of RAB38 may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of RAB38 may be introduced into a cell containing the target nucleic acid sequence, e.g., by formation of an polynucleotide-lipid complex, as described in WO 90/10448. It is understood that the use of antisense molecules or knock out and knock in models may also be used in screening assays as, in addition to methods of treatment.

Intrabodies also provide effective means for blocking or modulating the activity of RAB38 proteins. The intracellularly expressed antibody constructs, usually single-chain Fv antibodies, are directed against a target inside a cell. The scFv gene can be transferred into cells, where scFv protein expression can modulate the properties of its target, e.g. RAB38, sometimes extinguishing protein function and causing a phenotypic knockout.

Topical Administration of RAB38 Inhibitors

To be effective, a topical, RAB38 modulator or inhibitor should contain sufficient concentration of the active agent to produce an modulatory effect, where preferably the modulatory effect is an inhibitory effect, it should penetrate the tissue such as intact skin sufficiently to deliver a therapeutic dose, and it should exhibit rapid onset of action and have a prolonged effect. In achieving the foregoing, it is often desirable to have the RAB38 modulator present in a high concentration to effect a rapid onset and, additionally or alternatively, in excess of the amount that can be immediately absorbed through the dermis at the site of application, so as to prolong RAB38 modulation.

In the preferred topical formulations of the invention, the active RAB38 modulator is one which is generally suited to topical drug administration and includes any such materials known in the art. The topical carrier is selected so as to provide the composition in the desired form, e.g., as a liquid, lotion, cream, paste, gel, powder, or ointment, and may be comprised of a material of either naturally occurring or synthetic origin. It is essential, clearly, that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. The composition of the invention may also be administered in the form of a shampoo, in which case conventional components of such a formulation are included as well, e.g., surfactants, conditioners, viscosity modifying agents, humectants, and the like.

Particularly preferred formulations herein are colorless, odorless ointments, lotions, creams and gels.

Ointments are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy for further information.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. A particularly preferred lotion formulation for use in conjunction with the present invention contains propylene glycol mixed with a hydrophilic petrolatum such as that which may be obtained under the trademark Aquaphor.RTM. from Beiersdorf, Inc. (Norwalk, Conn.).

Creams containing the selected RAB38 modulator are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant The emulsifier in a cream formulation, as explained in Remington, supra, is generally a nonionic, anionic, cationic or amphoteric surfactant.

Gel formulations are may also be used. As will be appreciated by those working in the field of topical drug formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Various additives, known to those skilled in the art, may be included in the topical formulations of the invention. For example, solvents may be used to solubilize certain drug substances. Other optional additives include skin permeation enhancers, opacifiers, anti-oxidants, gelling agents, thickening agents, stabilizers, and the like. Other agents may also be added, such as antimicrobial agents, antifungal agents, antibiotics and anti-inflammatory agents such as steroids.

In the preferred topical formulations of the invention, the active RAB38 modulator is present in an amount which is generally less than 10% by weight of the total composition, preferably less than about 1% by weight, and most preferably less than about 0.1% by weight. is present in an amount which is generally less than 10% by weight of the total composition, preferably less than about 1% by weight, and most preferably less than about 0.1% by weight.

### DEFINITIONS

To facilitate understanding of the invention, a number of terms are defined and discussed below.

The term “therapeutically effective amount” is intended to mean the amount of RAB3 8 modulator sufficient to produce an effect when applied topically. These amounts are known in the art or may be determined by methods known in the art, and typically range from about 1 to 20,000 mg per human adult and preferably about 10 to 10,000 mg and most preferably range from about 20 to 5,000 mg of the RAB38 modulatory agent per application, depending upon the RAB38 modulator chosen, and whether the tissue, such as the skin is the site of action.

The term “inhibitor of melanosme pigmentation” refers to any substance that blocks to any degree the formation of melanosomes and/or which blocks to any degree the deposition of pigment within the melanosomes. The inhibitor may act at any step of the processes leading to melanosome pigmentation including but not limited to, trafficking of melanosome vesicles, sorting of vesicles or proteins, or effects on protein activity.

The term “lightening of skin color” refers to a visible lessening of color, tone or shade of the skin, however slight. The change may be brought about by decreases in pigmentation of the melanosomes that arise as a result of reduced or inhibited RAB38 function.

“Inhibitors,” “activators” or “modulators” of RAB38 polypeptide refer to inhibitory or activating molecules identified using in vitro and in vivo assays for RAB38 activity. Inhibitors are compounds that decrease, block, prevent, delay activation, inactivate, desensitize, or down regulate the activity. Activators are compounds that increase, open, activate, facilitate, enhance activation, sensitize or up regulate RAB38 activity. Such assays for inhibitors and activators include e.g., expressing a RAB38 polypeptide in a cell and then measuring GTPase activity. Alternatively, cells expressing endogenous RAB38 can be used in such assays. To examine the extent of inhibition, samples or assays comprising a RAB38 protein are treated with a potential activator or inhibitor and are compared to control samples without the inhibitor. Control samples (untreated with inhibitors) are assigned a relative RAB38 activity value of 100%. Inhibition of RAB38 activity is achieved when the RAB38 activity value relative to the control is about 90%, preferably 50%, more preferably 25-0%. Activation of RAB38 activity is achieved when the RAB38 activity value relative to the control is 110%, more preferably 150%, most preferably at least 200-500% higher or 1000% or higher.

The term “intrabody” or “intrabodies” refers to intracellularly expressed antibody constructs, usually single-chain Fv antibodies, directed against a target inside a cell. Nam, C H, et al. (2002) Methods Mol Biol. 193:301; der Maurr, A A, et al. (2002) J. Biol Chem November 22; 277(47):45075; Cohen, P A (2002) Methods Mol Biol 178:367. The scFv gene can be transferred into cells, where scFv protein expression can modulate the properties of its target, e.g. RAB38, sometimes extinguishing protein function and causing a phenotypic knockout. Indeed, the scFv intrabody can be expressed in the cytoplasm and directed to any cellular compartment where it can target intracellular proteins and elicit specific biological effects. Intrabodies thus provide effective means for blocking or modulating the activity of proteins.

The term “ribozyme” refers an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto, et al. (1994) Adv. in Pharmacology 25: 289-317 for a general review of the properties of different ribozymes).

The general features of hairpin ribozymes are described, e.g., in Hampel, et al. (1990) Nucl. Acids Res. 18:299-304; European Patent Publication No. 0 360 257; U.S. Pat. No. 5,254,678. Methods of preparation are described in, e.g., WO 94/26877; Ojwang, et al. (1993) Proc. Natl. Acad. Sci. USA 90:6340-6344; Yamada, et al. (1994) Human Gene Therapy 1:39-45; Leavitt, et al. (1995) Proc. Natl. Acad. Sci. USA 92:699-703; Leavitt, et al. (1994) Human Gene Therapy 5:1151-120; and Yamada, et al. (1994) Virology 205: 121-126.

Any cellular gene product expressed as RNA, including proteins encoded by mRNA and structural RNAs themselves, can be targeted for specific cleavage and inactivation by ribozymes engineered to include appropriate regions of sequence and structure. New ribozymes have been engineered from the group I intron, either by directed evolution or by careful rational design, that cleave alternate substrates, including a DNA substrate.

The term “siRNA” refers to a small interfering RNA molecule which is part the RNA interference mechanism. RNA interference is a mechanism to suppress gene expression in a sequence specific manner. See, e.g., Brumelkamp, et al. (2002) Sciencexpress (21 Mar. 2002); Sharp (1999) Genes Dev. 13:139-141; and Cathew (2001) Curr. Op. Cell Biol. 13:244-248. In mammalian cells, short, e.g., 21 nt, double stranded small interfering RNAs (siRNA) have been shown to be effective at inducing an RNAi response. See, e.g., Elbashir, et al. (2001) Nature 411:494-498. The mechanism may be used to downregulate expression levels of identified genes, e.g., to inhibit of RAB38 function for the purpose of decreasing skin pigmentation.

The terms “antisense molecule”, “antisense oligonucleotide specific for RAB38”, or “RAB38 antisense oligonucleotide” is an oligonucleotide having a sequence (i) capable of forming a stable triplex with a portion of a RAB3 8 nucleotide sequence, or (ii) capable of forming a stable duplex with a portion of an mRNA transcript of AB38.

Antisense technology is emerging as an effective means of lowering the levels of a specific gene product. It is based on the findings that these “antisense” sequences hybridize a gene or associated target polynucleotide, to form a stable duplex or triplex, based upon Watson-Crick or Hoogsteen binding, respectively. The specifically bound antisense compound then either renders the respective targets more susceptible to enzymatic degradation, blocks translation or processing, or otherwise blocks or inhibits the function of a target polynucleotide. Where the target polynucleotide is RNA, the antisense molecule hybridizes to specific RNA transcripts disrupting normal RNA processing, stability, and translation, thereby preventing the expression of a targeted gene. Administration of antisense oligonucleotides or transfer of expression constructs capable of producing intracellular antisense sequences complementary to the mRNA of interest have been shown to block the translation of specific genes in vitro and in vivo. For example, Holt et al., Mol. Cell. Biol. 1988, 8, 963-973, focusing on c-myc, found the formation of an intra-cellular duplex with target mRNA and a selective decrease of c-myc protein in human promyelocytic leukemia HL-60 cells.

The term “oligonucleotide” as used herein refers to a polynucleotide formed from joined nucleotides. Moreover, the term “oligonucleotide” includes naturally occurring oligonucleotides or synthetic oligonucleotides formed from naturally occurring subunits or analogous subunits designed to confer special properties on the oligonucleotide so that it is more stable in biological systems or binds more tightly to target sequences. It also includes modifications of the oligonucleotides such as chemically linking them to other compounds that will enhance delivery to cells or to the nucleus and other compartments of cells. Further, oligonucleotides of the invention may contain modified internucleotide linkages to provide stability against nucleases. For example, the invention may include phosphorothioate oligodeoxyribonucleotides. Thus, the term “oligonucleotide” includes unmodified oligomers of ribonucleotides and/or deoxyribonucleotides, as well as oligomers wherein one or more purine or pyrimidine moieties, sugar moieties or internucleotide linkages is chemically modified.

Without limiting the generality of the foregoing, the term “oligonucleotide” as used herein includes linear oligomers of natural or modified monomers or linkages, including deoxyribonucleosides, ribonucleosides, .alpha.-anomeric forms thereof, polyamide nucleic acids, and the like, capable of specifically binding to a target polynucleotide by way of a regular pattern of monomer-to-monomer interactions, such as Watson-Crick type of base pairing, Hoogsteen or reverse Hoogsteen types of base pairing, or the like. Usually, monomers are linked by phosphodiester bonds or analogs thereof to form oligonucleotides ranging in size from a few monomeric units, e.g., 3-4, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate, and the like, as more fully described below. As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g., described generally by Scheit, Nucleotide Analogs (John Wiley, New York, 1980). Such analogs include synthetic nucleosides designed to enhance binding properties, e.g., duplex or triplex stability, specificity, or the like. It is preferred that the oligonucleotides of the invention be modified to increase stability and prevent intracellular and extracellular degradation. It is more preferred that the oligonucleotides of the invention be modified to increase their affinity for target sequences, and their transport to the appropriate cells and cell compartments when they are delivered into a mammal in a pharmaceutically active form.

Target polynucleotides may be single-stranded or double-stranded RAB38 DNA or RAB38 RNA; however, single-stranded DNA or RNA targets are preferred. It is understood that the target to which the RAB38 antisense oligonucleotides of the invention are directed include allelic forms of RAB38. There is substantial guidance in the literature for selecting particular sequences for antisense oligonucleotides given a knowledge of the sequence of the target polynucleotide, e.g., Peyman and Ulmann, Chemical Reviews, 90:543-584, 1990; Crooke, Ann. Rev. Pharmacal. Toxicol., 32:329-376 (1992); and Zamecnik and Stephenson, Proc. Natl. Acad. Sci., 75:280-284 (1974). Preferably, the sequences of RAB38 antisense compounds are selected such that the G-C content is at least 60%. Preferred proto-oncogene mRNA targets include the 5′cap site, tRNA primer binding site, the initiation codon site, the mRNA donor splice site, and the mRNA acceptor splice site, e.g., Goodchild et al., U.S. Pat. No. 4,806,463.

Antisense oligonucleotides of the invention may comprise any polymeric compound capable of specifically binding to a target polynucleotide by way of a regular pattern of monomer-to-nucleoside interactions, such as Watson-Crick type of base pairing, Hoogsteen or reverse Hoogsteen types of base pairing, or the like. Antisense compounds of the invention may also contain pendent groups or moieties, either as part of or separate from the basic repeat unit of the polymer, to enhance specificity, nuclease resistance, delivery, or other property related to efficacy, e.g., cholesterol moieties, duplex intercalators such as acridine, poly-L-lysine, “end-capping” with one or more nuclease-resistant linkage groups such as phosphorothioate, and the like.

For example, it is known that enhanced lipid solubility and/or resistance to nuclease digestion results by substituting an alkyl group or alkoxy group for a phosphate oxygen in the internucleotide phosphodiester linkage to form an alkylphosphonate oligonucleoside or alkylphosphotriester oligonucleotide. Non-ionic oligonucleotides such as these are characterized by increased resistance to nuclease hydrolysis and/or increased cellular uptake, while retaining the ability to form stable complexes with complementary nucleic acid sequences. The alkylphosphonates, in particular, are stable to nuclease cleavage and soluble in lipid. The preparation of alkylphosphonate oligonucleosides is disclosed in Tso et al., U.S. Pat. No. 4,469,863.

Preferably, nuclease resistance is conferred on the antisense compounds of the invention by providing nuclease-resistant internucleosidic linkages. Many such linkages are known in the art, e.g., phosphorothioate: Zon and Geiser, Anti-Cancer Drug Design, 6:539-568 (1991); Stec et al., U.S. Pat. No. 5,151,510; Hirschbein, U.S. Pat No. 5,166,387; Bergot, U.S. Pat. No. 5,183,885; phosphorodithioates: Marshall et al., Science, 259:1564-1570 (1993); Caruthers and Nielsen, International application PCT/US89/02293; phosphoramidates, e.g., —OP (.dbd.O) (NR.sup.1 R.sup.2)-O— with R.sup.1 and R.sup.2 hydrogen or C.sub.1 -C.sub.3 alkyl; Jager et al., Biochemistry, 27:7237-7246 (1988); Froehler et al., International application PCT/US90/03138; peptide nucleic acids: Nielsen et al., Anti-Cancer Drug Design, 8: 53-63 (1993), International application PCT/EP92/01220; methylphosphonates: Miller et al., U.S. Pat. No. 4,507,433, Ts'o et al., U.S. Pat No. 4,469,863; Miller et al., U.S. Pat. No. 4,757,055; and P-chiral linkages of various types, especially phosphorothioates, Stec et al., European patent application 506,242 (1992) and Lesnikowski, Bioorganic Chemistry, 21:127-155 (1993). Additional nuclease linkages include phosphoroselenoate, phosphorodiselenoate, phosphoroanilothioate, phosphoranilidate, alkylphosphotriester such as methyl- and ethylphosphotriester, carbonate such as carboxymethyl ester, carbamate, morpholino carbamate, 3′-thioformacetal, silyl such as dialkyl (C.sub.1-C.sub.6)- or diphenylsilyl, sulfamate ester, and the like. Such linkages and methods for introducing them into oligonucleotides are described in many references, e.g., reviewed generally by Peyman and Ulmann, Chemical Reviews 90:543-584 (1990); Milligan et al., J. Med. Chem., 36:1923-1937 (1993); Matteucci et al., International application PCT/US91/06855.

Resistance to nuclease digestion may also be achieved by modifying the internucleotide linkage at both the 5′ and 3′ termini with phosphoroamidites according to the procedure of Dagle et al., Nucl. Acids Res. 18, 4751-4757 (1990).

Preferably, phosphorous analogs of the phosphodiester linkage are employed in the compounds of the invention, such as phosphorothioate, phosphorodithioate, phosphoramidate, or methylphosphonate. More preferably, phosphorothioate is employed as the nuclease resistant linkage.

Phosphorothioate oligonucleotides contain a sulfur-for-oxygen substitution in the internucleotide phosphodiester bond. Phosphorothioate oligonucleotides combine the properties of effective hybridization for duplex formation with substantial nuclease resistance, while retaining the water solubility of a charged phosphate analogue. The charge is believed to confer the property of cellular uptake via a receptor (Loke et al., Proc. Natl. Acad. Sci., 86, 3474-3478 (1989)).

It is understood that in addition to the preferred linkage groups, compounds of the invention may comprise additional modifications, e.g., boronated bases, Spielvogel et al., U.S. Pat. No. 5,130,302; cholesterol moieties, Shea et al., Nucleic Acids Research, 18:3777-3783 (1990) or Letsinger et al., Proc. Natl. Acad. Sci., 86:6553-6556 (1989); and 5-propynyl modification of pyrimidines, Froehler et al., Tetrahedron Lett., 33:5307-5310 (1992).

Oligonucleotides of the invention may be synthesized by any method known in the art. It is preferred in the present invention that the oligonucleotides be prepared using synthetic chemical methods, such as, for example, phosphoramidite chemistry by sulfurization with tetraethylthiuram disulfide in acetonitrile. See, for example, Vu and Hirschbein, Tetrahedron Lett. 1991, 32, 30005-30008. Oligonucleotides of the invention may also be synthesized using in vitro and in vivo transcription systems, such as transcription by T.sup.7 polymerase or expression vectors. Oligonucleotides synthesized using in vitro and in vivo transcription systems may be modified via chemical methods known to those skilled in the art. Examples of such modifications include encapsulation in liposomes, or chemical linkage to steroids, antibodies, and cell receptor ligands.

In embodiments where triplex formation is desired, there are constraints on the selection of target sequences. Generally, third strand association via Hoogsteen type of binding is most stable along homopyrimidine-homopurine tracks in a double stranded target. Usually, base triplets form in T-A*T or C-G*C motifs (where “-” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 258:1463-1466 (1992); Distefano et al., Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267: 5712-5721 (1992); Yoon et al., Proc. Natl. Acad. Sci., 89:3840-3844 (1992); and Blume et al., Nucleic Acids Research, 20:1777-1784 (1992).

The length of the oligonucleotide moieties is sufficiently large to ensure that specific binding will take place only at the desired target polynucleotide and not at other fortuitous sites, as explained in many references, e.g., Rosenberg et al., International application PCT/US92/05305; or Szostak et al., Meth. Enzymol, 68:419-429 (1979). The upper range of the length is determined by several factors, including the inconvenience and expense of synthesizing and purifying large oligomers, the greater tolerance of longer oligonucleotides for mismatches than shorter oligonucleotides, whether modifications to enhance binding or specificity are present, whether duplex or triplex binding is desired, and the like.

It is preferred that the length of the oligonucleotide be between 5 and 200 nucleotides. It is more preferred that the oligonucleotide be between 10 and 50 nucleotides in length. It is most preferred that the oligonucleotide be between 15 and 25 nucleotides in length. In preferred embodiments, the oligonucleotide is specifically hybridizable with a translation initiation site. In one preferred embodiment of the present invention the oligonucleotide has the sequence 5′ AACGTTGAGGGGCAT 3′ (SEQ ID NO: 1). This oligonucleotide is complementary to a segment of the RAB38 mRNA beginning with a translation initiation codon and extending downstream therefrom

In general, the antisense oligonucleotides used in the practice of the present invention will have a sequence which is completely complementary to a selected portion of the target polynucleotide. Absolute complementarity is not however required, particularly in larger oligomers. Thus, reference herein to a “nucleotide sequence complementary to” a target polynucleotide does not necessarily mean a sequence having 100% complementarity with the target segment. In general, any oligonucleotide having sufficient complementarity to form a stable duplex with the target (e.g. the RAB38 mRNA) that is, an oligonucleotide which is “hybridizable”, is suitable. Stable duplex formation depends on the sequence and length of the hybridizing oligonucleotide and the degree of complementarity with the target polynucleotide. Generally, the larger the hybridizing oligomer, the more mismatches may be tolerated. One skilled in the art may readily determine the degree of mismatching which may be tolerated between any given antisense oligomer and the target sequence, based upon the melting point, and therefore the thermal stability, of the resulting duplex.

Preferably, the thermal stability of hybrids formed by the antisense oligonucleotides of the invention are determined by way of melting, or strand dissociation, curves. The temperature of fifty percent strand dissociation is taken as the melting temperature, T.sub.m, which, in turn, provides a convenient measure of stability. T.sub.m measurements are typically carried out in a saline solution at neutral pH with target and antisense oligonucleotide concentrations at between about 1.0-2.0 .mu.M. Typical conditions are as follows: 150 mM NaCl and 10 mM MgCl.sub.2 in a 10 mM sodium phosphate buffer (pH 7.0) or in a 10 mM Tris-HCl buffer (pH 7.0). Data for melting curves are accumulated by heating a sample of the antisense oligonucleotide/target polynucleotide complex from room temperature to about 85-90.degree. C. As the temperature of the sample increases, absorbance of 260 nm light is monitored at 1.degree. C. intervals, e.g., using a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of antisense oligonucleotides of different lengths and compositions.

According to one embodiment, the oligonucleotides of this invention are designed to be hybridizable with messenger RNA derived from the RAB38 gene. Such hybridization, when accomplished, interferes with the normal roles of the messenger RNA to cause a modulation of its function in the cell. The functions of messenger RNA to be interfered with include all vital functions such as translocation of the RNA to the site for protein translation, actual translation of protein from the RNA, splicing of the RNA to yield one or more mRNA species, and possibly even independent catalytic activity which may be engaged in by the RNA. The overall effect of such interference with the RNA function is to modulate expression of the RAB38 gene. By modulating the expression of RAB38, melanocyte pigmentation is modulated, or inhibited.

The terms “nucleic acid,” “nucleic acid sequence,” and “nucleotide sequence,” as used herein refer to an oligonucleotide or polynucleotide, and fragments or portions thereof, and to DNA or RNA of genomic or synthetic origin which can be single or double stranded, and represent the sense or antisense strand.

As used herein, the terms “restriction endonuclease” and “restriction enzyme” refer to bacterial enzymes, each of which cut double stranded DNA at or near a specific nucleotide sequence, referred to as a “restriction site.”

As used herein, the terms “complementary” or “complementarity” are used in reference to antiparallel polynucleotides (i.e., a sequence of nucleotides) related by the base pairing rules. For example, the sequence 5′ AGTTC-3′ is complementary to the sequence 3′-TCAAG 5′.

“Amplification” is defined as the production of additional copies of a nucleic acid sequence and is generally carried out using polymerase chain reaction (PCR) or other technologies well known in the art (e.g., Dieffenbach and Dveksler, PCR Primer, a Laboratory Manual, Cold Spring Harbor Press, Plainview N.Y. [1995]). As used herein, the term “polymerase chain reaction” (“PCR”) refers to the Mullis method for increasing the concentration of a segment of a target sequence in a mixture of genomic DNA without cloning or purification (See e.g., U.S. Pat. Nos. 4,683,195, 4,683,202 and 4,965,188, hereby incorporated by reference). This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension (DNA synthesis) are typically reiterated many times (i.e., denaturation, annealing and extension constitute one “cycle”; there usually are numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be VCR amplified.”

As used herein, the terms “PCR product” and “amplification product” refer to the resultant mixture of compounds after two or more cycles of the PCR steps of denaturation, annealing and extension are complete.

The terms “peptide,” “polypeptide” and “protein” all refer to a primary sequence of amino acids that are joined by covalent peptide linkages. In general, a peptide consists of a few amino acids, typically from 2-25 amino acids, and is shorter than a protein. “Polypeptides” encompass both peptides and proteins.

As used herein the term “portion” when in reference to a gene refers to fragments of that gene. In some embodiments, the fragments range in size from ten nucleic acids to the entire nucleic acid sequence minus one nucleic acid.

As used herein, the term “purify” or “purifying” refers to the removal of at least one contaminant from a sample. As used herein, the term “substantially purified” refers to molecules, either nucleic acids or amino acid sequences, that are removed from their natural environment, “isolated” or “separated,” and are largely free from other components.

The term “gene” refers to a nucleic acid (e.g., DNA) sequence comprised of parts, that when appropriately combined in either a native or recombinant manner, provide some product or function. In addition to the coding region of the nucleic acid, the term “gene” also encompasses the transcribed nucleotide sequences of the full-length mRNA adjacent to the 5′ and 3′ ends of the coding region. These noncoding regions are referred to as 5′ and 3′ untranslated sequences (5′ UT and 3′ UT). Both the 5′ and 3′ UT may serve regulatory roles, including translation initiation, post transcriptional cleavage and polyadenylation. In preferred embodiments, a mammalian “Rab38 gene” is provided.

In some embodiments, the “genomic” form of a gene contains the sequences of the transcribed mRNA, as well as other non coding sequences. “Introns” or “intervening sequences” are segments of a gene which are contained in the primary transcript (i.e., hetero-nuclear RNA, or hnRNA), but are spliced out to yield the processed mRNA form. Conversely, “exons” are the segments of a gene corresponding to the processed mRNA sequence.

The terms “in operable combination,” and “operably linked” when used in reference to nucleic acid herein are used to refer to the linkage of nucleic acid sequences in such a manner that a nucleic acid molecule capable of directing the transcription of a given gene and/or the synthesis of a desired protein molecule is produced. In preferred embodiments of the present invention, a mammalian Rab38 gene in “operable combination” with a promoter and/or an enhancer is provided.

As used herein, the term “vector” is used in reference to nucleic acid molecules that transfer DNA segments) from one cell to another. In some embodiments, a vector “backbone” comprises those parts of the vector which mediate its maintenance and enable its intended use (e.g., sequences necessary for replication, genes imparting drug or antibiotic resistance, a multiple cloning site, and possibly operably linked promoter/enhancer elements which enable the expression of a cloned nucleic acid). Vectors are often derived from plasmids, bacteriophages, or plant or animal viruses.

As used herein, the term “wild-type” refers to a gene or gene product which has the characteristics of that gene or gene product when isolated from a naturally occurring source. A wild-type gene is that which is most frequently observed in a population and is thus arbitrarily designated the “normal” or “wild-type” form of the gene.

In contrast, the terms “mutant” and “mutation” refer to a gene or gene product which displays modifications in sequence and or functional properties (i.e., altered characteristics) when compared to the wild type gene or gene product. It is noted that naturally-occurring mutants can be isolated and these are identified by the fact that they have altered characteristics when compared to the wild-type gene or gene product. In some embodiments, the present invention provides a mutant Rab38 gene or RAB38 protein. In preferred embodiments, the chocolate Rab38/RAB38 mutant is provided.

The term “candidate agent” refers to any molecule of any composition, including proteins, peptides, nucleic acids, lipids, carbohydrates, organic molecules, inorganic molecules, and/or combinations of molecules which are suspected to be capable of producing a physiological or biological response.

As used herein, the term “modulate” refers to a change in the activity of RAB38. For example, modulation may cause an increase or a decrease in enzymatic activity, binding characteristics, or any other biological, or functional properties of RAB38.

The term “melanocyte” as used herein, refers to special cells in the skin and the eye that synthesize melanin pigments. Clusters of melanocytes often appear on the skin as moles. The term “melanosome” refers to the melanin producing organelle of melanocytes.

As used herein, the terms “GTPase activity” and “guanosine triphosphatase activity” refer to the enzyme activity that hydrolyses GTP to produce GDP and orthophosphate. GTPase activity is regulated by GTPase activating proteins (activation) and by guanine nucleotide releasing proteins (inhibition). In the context of the invention, GTPase activity refers to “RAB38 activity” or the GTPase activity of RAB38. RAB38 is inactive when bound to GDP, and active when bound to GTP. Thus, the term “GTP binding” refers to the binding of GTP by a GTPase (e.g., RAB38), while the term “GDP release” refers to the release of GDP by a GTPase (e.g., RAB38). GTPases of the RAB family have been implicated in the process of vesicle trafficking. In the context of the invention, the term “RAB activity” encompasses “RAB38 trafficking” or the transport of RAB38 to melanosomes of melanocytes, and “TYRP1 trafficking” or the transport of TYRP1 to melanosomes.

As used herein the term “intradermal-penetration agent” means an agent capable of transporting a pharmacologically active compound through the stratum corneum and into the epidermis or dermis, while keeping the pharmacological effects restricted to the intracutaneous regions of drug penetration, preferably, with little or no systemic absorption.

A “penetration enhancing amount” of an intradermal-penetration agent is an amount which enhances the RAB38 inhibitor or modulator penetration rate through the stratum corneum, relative to the penetration rate without the intradermal-penetration agent.

The term “pharmaceutically acceptable topical formulation” as used herein means any formulation which is pharmaceutically acceptable for topical administration of a RAB38 modulator or inhibitor by application of the formulation to the epidermis. According to the invention, a “topical formulation” will comprise at least one RAB38 modulator or inhibitor and may also include an intradermal-penetration agent. The choice of topical formulation will depend or several factors, including the skin condition to be treated, c and other excipients present, their stability in the formulation, available manufacturing equipment, and costs constraints.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); BSA (bovine serum albumin); DMEM ( Dulbecco's modified eagle's medium); FBS (fetal bovine serum); H₂0 (water); aa (amino acid); bp (base pair); kb (kilobase pair); EST (expressed sequence tag); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); MW (molecular weight); sec (seconds); min(s) (minute/minutes); hr(s) (hour/hours); CO₂ (carbon dioxide); Cy3 (indocarbocyanine); Cy5 (indodicarbocyanine); (DEPC (diethyl pyrocarbonate); dNTPs (deoxynucleotides); MgCl₂ (magnesium chloride); PBS (phosphate buffered saline [150 mM NaCl, 10 mM sodium phosphate buffer, pH 7.2]); SDS (sodium dodecyl sulfate); SSC (saline sodium citrate buffer); TBS (Tris buffered saline); PCR (polymerase chain reaction); RT (reverse transcription); w/v (weight to volume); v/v (volume to volume); Applied Biosystems (Applied Biosystems, Foster City, Calif.); Axon (Axon Instruments Inc., Foster City, Calif.); GeneCodes (GeneCodes, Ann Arbor, Mich.); Invitrogen Invitrogen Corp., Carlsbad, Calif.); Jackson (Jackson Laboratory, Bar Harbor, Me.); NEB (New England Biolabs, Inc., Beverly, Mass.); NIH (National Institutes of Health, Bethesda, Md.); Qiagen (Qiagen, Valencia, Calif.); Roche (Roche, MannHeim, Germany); and Signet (Signet Laboratories, Dedham, Mass.).

### EXAMPLE 1

### Cell Culture

All cells for microarray analysis were grown to 90% confluence at 37° C. in 5% CO₂. The cell lines used in these experiments were obtained from Jeffry Trent (NIH). Melanoma cell lines were grown in RPMI media containing 10% FBS, 2 mM L glutamine and 100 units/ml each of penicillin and streptomycin. Media for MnSOD6 cl1 also contained 500 μg/ml geneticin. 293T, U138 and HeLa cells were grown in DMEM media containing 10% FBS, 2 mM L glutamine and 100 units/ml each of penicillin and streptomycin. Primary murine melanocytes were cultured as previously described (Wu et al., J Cell Sci 114:1091-1100 [2001]. Melan-a cells were cultured in RPMI 1640 media containing 10% FBS, 2 mM L-glutamine, 10 mM sodium pyruvate, 100 units/ml each of penicillin and streptomycin, 200 mm 12-O-tetradecanoylphorbol 13-acetate, 0.01 mM sodium bicarbonate and 0.1 mM 2-mercaptoethanol in 5% C0₂.

### EXAMPLE 2

### RNA Preparation

Cells for microarray analysis were obtained in pools of four 500 cm² dishes, that were harvested by scraping, washed in PBS, and pelleted. Pellets were lysed in 10 ml Trizol reagent (Invitrogen). Two ml of chloroform were added, the sample was shaken, and then centrifuged to separate phases. The aqueous layer was removed and an equal volume of 75% ethanol was added dropwise while vortexing. Sample was applied to a RNeasy maxi column (Qiagen) and the manufacturer's purification protocol was followed. Samples were eluted in water, precipitated with 3 M sodium acetate and stored at −80° C. RNA pellets were resuspended in DEPC water to 1 μg/p,l concentration, and applied to a Microcon 100 column. RNA samples were centrifuged and concentrated to 7 10 μg/ul.

### EXAMPLE 3

### Labeling and Hybridization

RNA was reverse transcribed to fluorescent labeled cDNA and co hybridized on slides in experimental/reference pairs. Expressed sequence tag (EST) clone inserts were prepared and applied to slides as described (DeRisi et al., Nat Genet 14:457-460 [1996]). Reversed transcribed (RT) fluorochrome labeled cDNA was generated as known in the art (See, The National Human Genome Research Initiative's Microarray Project web site). For reactions, 60 μg of total RNA (Cy3) or 120 μg of total RNA (Cy5) were used. Hybridizations were carried out in a final volume of 40 μl at 65° C. in a humidified chamber for 16 hr. Slides were washed at room temperature in 0.5×SSC/0.1% SDS for 3 min followed by a second wash in 0.6×SSC for 3 min. Slides were immediately spun dry by centrifugation.

### EXAMPLE 4

### Image Acquisition and Analysis

Fluorescence signal intensities for Cy3 (532 λ) and Cy5 (635 λ) fluorochromes were obtained using a Genepix 4000a scanner (Axon) at 10 μM resolution. A set of 88 housekeeping control genes was used to normalize for labeling efficiency (Loftus et al., Proc Natl Acad Sci USA 96:9277-9280 [1999]). Expression profile analysis was performed with a clustering algorithm using average linkage method and Pearson's correlation similarity measurement (See, The National Human Genome Research Initiative's Genome Clustering web site).

### EXAMPLE 5

### Organization of the Mouse Rab38 Gene

To facilitate mutation screening of the mouse Rab38 gene, database searches were employed to identify genomic DNA adjacent to Rab38 coding sequences. Rab38 mRNA sequence (SEQ ID NO:9 and GenBank Accession No. AY062237), was BLASTed against mouse genomic sequencing trace archives (See, The National Center for Biotechnology Information's web site for Trace Archive Querying). Significant similarities were determined by a returned BLAST score of greater than 200. Relevant data were downloaded and aligned to the mRNA sequence using Sequencher version 3.1.1 (GeneCodes). Genomic organization was confirmed by using the Spidey program (See, The National Center for. Biotechnology Information's web site for Spidey). Gene organization was also experimentally confirmed through PCR and DNA sequencing of genomic fragments. This analysis revealed that Rab38 is composed of three exons: exon 1, nucleotides 1-292; exon 2, nucleotides 292-572; and exon 3, nucleotides 573-1439 (nucleotide positions refer to GenBank Accession No. AY062237). The confirmed exon and surrounding intron sequences have been deposited into GenBank as: exon 1, AF448441 (SEQ ID NO:10); exon 2, AF448442 (SEQ ID NO: 11); and exon 3, AF448443 (SEQ ID NO:12).

### EXAMPLE 6

### In Situ Hybridization

Timed matings were used to obtain staged FVBINJ (Jackson) mouse embryos, and EO.5 was designated as noon on the day of vaginal plug formation. Embryos were fixed overnight in 4% paraformaldehyde in PBS. Digoxigenin conjugated probes were synthesized by reverse transcription (RT) of linearized plasmids and/or PCR products with RT binding site linkers (Roche). The following DNA sources were used for probe synthesis: Tyrosinase, cDNA clone 4633402007; Tyrp1, RT-PCR from B16 cell line total RNA (TYRP15′T3F 5′-GCGCGAATTA ACCCTCACTA AAGGGTCTGA GCACCCCTGT CTTCT 3′, SEQ ID NO:13; TYRP15′T7R 5′-GCGCGTAATACGACTCACTA TAGGGCCCAG TTGCAAAATT CCAGT-3′, SEQ ID NO:14); Dct, cDNA; Aiml/Matp, RIKEN cDNA clone G370045L22; Mlsnl RT-PCR from B16 cell line total RNA (MLSN R T7 5′ GCGGGTAATA CGACTCACTA TAGGGGCCAC AAACATGTCC TACTTAC-3′, SEQ ID NO:15; MLSN FT3 5′ GCGCGAATTA ACCCTCACTA AAGGGAAGCT TCCGGACTCT CTAC 3′, SEQ ID NO:16); Rab38, Riken cDNA clone 23 10011 F14. In situ hybridizations were performed using published protocols (Wilkinson and Nieto, Methods Enzymol 225:361-373 [1993]), with the following modifications. After probe hybridization, Ribonuclease A digestion was omitted, TBS was used in place of PBS, and the substrate BM-purple (Roche) was used in place of 5-bromo4 chloro-3-indolyl phosphate/nitro blue tetrazolium.

### EXAMPLE 7

### Mutation Detection

The mouse Rab38 gene homologue was screened for mutations by PCR amplification and DNA sequencing of exon containing genomic segments. The following primer pairs were designed to amplify the three protein coding exons, as well as a small amount of flanking intron DNA: Rab38 Ex1F 5′-TAGGAAGGAGGATTAAACCC G 3′ (SEQ ID NO:17) and Rab38 Ex 1R 5′ GAACTCCTCATGGCTCACTC C 3′ (SEQ ID NO:18) yielding a 428 bp product; Rab38 Ex2F 5′-GGATATGAAGCTCCAGTGTA GTGTAC 3′ (SEQ ID NO:19) and Rab38 Ex2R 5′CACTGGACAG AAACATTATT GTCAC 3′ (SEQ ID NO:20) yielding a 464 bp product; and Rab38 Ex3F 5′-AAGTTATCAG CCAGTGAGAT ACTGTG 3′ (SEQ ID NO:21) and Rab38 Ex3R 5′-CACATGTGGT ATATCTATCC TGACG 3′ (SEQ ID NO:22) yielding a 526 bp product. PCR reactions contained 1.5 μM of each primer, 0.2 μM dNTPs, 1.5 pM MgCl₂, 1 unit AmpliTaq DNA Polymerase (Applied Biosystems), 1× of the manufacturer's 1O× buffer, and 40 ng of mouse genomic DNA. Thermal cycling consisted of an initial denaturation for 2 min at 93° C., followed by 40 cycles of 93° C. for 10 sec, 55° C. for 5 sec, and 72° C. for 30 sec. A final extension at 72° C. was performed for 7 min. Following separation on a 1% agarose gel, PCR products were excised and purified using the QIAquick Gel Extraction Kit (Qiagen). DNA sequencing was performed with BigDye terminator chemistry, and a model 3100 DNA sequencing instrument (Applied Biosystems). The cycle-sequencing routine was 30 cycles of 92° C. for 20 sec, 55° C. for 10 sec, and 60° C. for 4 min with a 20 μL reaction containing 8 μL of BigDye cocktail, 0.5 μL of a 25 [tM primer solution, 6.5 μL of water, and 5 μL of PCR product (at ˜50 ng/μL). Data was extracted and analyzed using Sequencing Analysis version 3.3 (Applied Biosystems) and aligned with Sequencher software version 3.1.1 (GeneCodes). Alignments included sequence data derived from wildtype C57B 1/6J and heterozygous cht/+ mice, as well as mouse Rab38 mRNA sequence (GenBank Accession No. AY062237).

### EXAMPLE 8

### Mutation Confirmation and Genotyping

Primers were designed to amplify a 213 by fragment surrounding the G146T sequence change (cht Ex1F 5′-GGCCTCCAGG ATGCAGACAC C 3′, SEQ ID NO:23; cht Ex1R 5′ CCAGCAATGT CCCAGAGCTG C 3′, SEQ ID NO:24). PCR amplification was performed as described in Example 7. SexAI and BsaJI restriction digests were performed using 20 [tL restriction enzyme digests containing 10 μL of PCR product, 2.5 units enzyme (NEB) along with 1× of the supplied BSA and digest buffer. Reactions were incubated overnight at the manufacturer's suggested temperature and electrophoresed on a 2% agarose gel to visualize band patterns.

### Example 9

### Cell Transfection

GFP-RAB38 constructs were generated by PCR amplifying mouse Rab38 with att site linker primers (AttB1-RRab 5′-GGGGACAAGT TTGTACAAAA AAGCAGGCTC CATGCAGACA CCTCACAAG-′, SEQ ID NO:25 and AttB2-RRab-STP 5′-GGGGACCACT TTGTACAAGA AAGCTGGGTT CTAGGATTTG GCACAGCCAG A 3′, SEQ ID NO:26) and “Gateway38 cloning into pDest 53 (Invitrogen) as per manufacturer's instructions. GFP-RAB38 was transfected into melan-a cells using LipofectAMINE 2000 (Invitrogen) with a DNA/LipofectAMINE 2000 ratio of 1.6 g/4 μl in a 4 cm² surface area as per manufacturer's instructions. After 72 hours, cells were fixed and stained with a 1:200 dilution of the TYRP1-reactive antibody MEL5 (Signet) as previously described (Wu et al., J Cell Sci 110:847-859 [1997]).

### EXAMPLE 10

### Bleeding Times

Bleeding times were assayed in four C57B1/6J Rab38^{cht}/Rab38^{cht} and four C57B1/6J animals, as described in the art (Sviderskaya et al., Genetics 148:381-390 [1998]). Assayed mice were 6-12 weeks of age. A 2 mm portion of the tail was removed and the cut tail immediately immersed in saline at 37° C. Each mouse was maintained in a horizontal position in a restrainer with the tip of the tail held 4-5 cm below the body. Bleeding time was that required for the small stream of blood to stop abruptly.

All publications and patents mentioned in the above specification are herein incorporated by reference. Various modifications and variations of the described method and system of the invention will be apparent to those skilled in the art without departing from the scope and spirit of the invention Although the invention has been described in connection with specific preferred embodiments, it should be understood that the invention as claimed should not be unduly limited to such specific embodiments. Indeed, various modifications of the described modes for carrying out the invention which are obvious to those skilled in the art and/or related fields are intended to be within the scope of the present invention. pDest 53 Invitrogen) as per manufacturer's instructions. GFP RAB38 was transfected into melan a cells using LipofectAMINE 2000 (Invitrogen) with a DNA/LipofectAMINE 2000 ratio of 1.6 g/4 μl in a 4 cm′ surface area as per manufacturer's instructions. After 72 hours, cells were fixed and stained with a 1:200 dilution of the TYRP1 reactive antibody MELS (Signet) as previously described (Wu et al., J Cell Sci 110:847-859 [1997]).

What is claimed is:

1. An isolated nucleic acid comprising a sequence selected from the group consisting of SEQ ID NO:9, SEQ ID NO: 10, SEQ ID NO: 11, and SEQ ID NO:12.

2. The isolated nucleic acid of claim 1, wherein said nucleic acid is deoxyribonucleic acid.

3. An isolated nucleic acid, wherein said nucleic acid is the complement of the nucleic acid of claim 1.

4. A vector comprising the nucleic acid of claim 1.

5. A host cell comprising the vector of claim 4.

6. An isolated protein encoded by the nucleic acid sequence set forth in SEQ ID NO:9.

7. A method for detecting mutations in Rab38 comprising the steps of:
a) amplifying at least a portion of Rab38 from genomic DNA to yield a Rab38 amplification product;
b) purifying said Rab38 amplification product; and
c) sequencing said Rab38 amplification product.

8. The method of claim 7, wherein said amplifying is accomplished using a polymerase chain reaction.

9. The method of claim 7, wherein said at least a portion of Rab38 is selected from the group consisting of at least one Rab38 exon, at least one Rab38 intron, the Rab38 5′ untranslated sequence, and the Rab38 3′ untranslated sequence.

10. The method of claim 9, wherein said at least one Rab38 exon is selected from the group consisting of Rab38 exon 1, Rab38 exon 2, and Rab38 exon 3.

11. The method of claim 7, wherein said genomic DNA is mammalian genomic DNA.

12. The method of claim 7, wherein said purifying is accomplished using size selection.

13. A method for detecting mutations in Rab38 comprising the steps of:
a) amplifying at least a portion of Rab38 from genomic DNA to yield a Rab38 amplification product;
b) digesting said Rab38 amplification product to yield a digested Rab38 amplification product; and
c) electrophoresing said digested Rab38 amplification product.

14. The method of claim 13, wherein said amplifying is accomplished using a polymerase chain reaction.

15. The method of claim 13, wherein said at least a portion of Rab38 is selected from the group consisting of at least one Rab38 exon, at least one Rab38 intron, the Rab38 5′ untranslated sequence, and the Rab38 3′ untranslated sequence.

16. The method of claim 15, wherein said at least one Rab38 exon is selected from the group consisting of Rab38 exon 1, Rab38 exon 2, and Rab38 exon 3.

17. The method of claim 13, wherein said genomic DNA is mammalian genomic DNA.

18. A method for screening for biologically active agents to modulate RAB38 activity, comprising the steps of:
a) providing:
i) melanocytes comprising RAB38 activity, and
ii) a candidate agent; and
b) exposing said melanocytes to said candidate agent to yield treated melanocytes; and
c) measuring the modulation of said RAB38 activity of said treated melanocytes by said candidate agent.

19. The method of claim 18, wherein said RAB38 activity comprises GTPase activity.

20. The method of claim 18, wherein said RAB38 activity comprises GTP binding activity.

21. The method of claim 18, wherein said RAB38 activity comprises GDP release.

22. The method of claim 18, wherein said RAB38 activity comprises TYRP1 trafficking to melanosomes.

23. The method of claim 18, wherein said RAB38 activity comprises RAB38 trafficking to melanosomes.

24. A kit for screening for biologically active agents that modulate RAB38 activity, comprising: a) plurality of melanocytes comprising RAB38 activity, wherein said melanocytes are provided within a container, and b) instructions for determination of RAB38 activity in said melanocytes.

25. The kit of claim 24, further comprising means to analyze RAIB38 activity.

26. The kit of claim 25, wherein said means to analyze RAB38 activity comprises an assay to assess GTPase activity.

27. The kit of claim 25, wherein said means to analyze RAB38 activity comprises an assay to assess GTP binding activity.

28. The kit of claim 25, wherein said means to analyze RAB38 activity comprises an assay to assess GDP release.

29. The kit of claim 25, wherein said means to analyze RAB38 activity comprises an assay to assess TYRP 1 trafficking to melanosomes.

30. The kit of claim 25, wherein said means to analyze RAB38 activity comprises an assay to assess RAB38 trafficking to melanosomes.

31. A kit for detection of mutations in RAB38 comprising at least two primer sequences suitable for amplification of at least a portion of RAB38, and instructions for utilizing said kit.

32. The kit of claim 31, wherein said primer sequences are selected from the group consisting of SEQ ID NO:17, SEQ ID NO:18, SEQ ID NO:19, SEQ ID NO:20, SEQ ID NO:21, and SEQ ID NO:22.

33. The kit of claim 31, wherein said kit is suitable for use in the polymerase chain reaction.

34. The kit of claim 31, further comprising reagents for digesting nucleic acid.

35. A kit for diagnosing defects in melanosome function, comprising melanocytes comprising RAB38 and instructions for assessing defects in melanosome function.

36. The kit of claim 35, further comprising means to analyze RAB38 activity.

37. The kit of claim 36, wherein said means to analyze RAB38 activity comprises an assay to assess GTPase activity.

38. The kit of claim 36, wherein said means to analyze RAB38 activity comprises an assay to assess GTP binding activity.

39. The kit of claim 36, wherein said means to analyze RAB38 activity comprises an assay to assess GDP release.

40. The kit of claim 36, wherein said means to analyze RAB38 activity comprises an assay to assess TYRP1 trafficking to melanosomes.

41. The kit of claim 36, wherein said means to analyze RAB38 activity comprises an assay to assess RAB38 trafficking to melanosomes.

42. A composition for modulating pigmentation of melanocytes, comprising a modulator of RAB38 activity.

43. The composition of claim 42, wherein the modulator of RAB38 activity is an enhancer of RAB38 activity.

44. The composition of claim 42, wherein the modulator of RAB38 activity is an inhibitor of RAB38 activity.

45. The composition of claim 44, wherein the inhibitor of RAB38 activity is selected from the group consisting of siRNA and intrabodies.

46. A method of modulating the pigmentation of a melanosome and changing skin color, the method comprising: contacting a skin surface with a modulator of RAB38 activity, thereby regulating the activity of RAB38.

47. The method of claim 46, wherein the modulator is an inhibitor of RAB38 activity that down-regulates RAB38 activity and lightens skin color.

48. The method of claim 46, wherein the modulator is an enhancer of RAB38 activity that up-regulates RAB38 activity and darkens skin color.


SEQUENCE LISTING (28 sequences)

SEQ ID NO: 1 - DNA, 8 bp, Mus musculus
  Rab38 sequence of wildtype allele in C57Bl6/J +/+ DNA
ctgggtgt

SEQ ID NO: 2 - DNA, 8 bp, Mus musculus
  Rab38 sequence of chocolate (cht) mutant allele in c57Bl6/J Rab38cht/+ DNA
ctggktgt

SEQ ID NO: 3 - protein, 34 aa, Homo sapiens
  human RAB38 highly conserved N-terminal region
MQAPHKEHLYKLLVIGDLGVGKTSIIKRYVHQNF

SEQ ID NO: 4 - protein, 34 aa, Rattus norvegicus
  rat RAB38 highly conserved N-terminal region
MQTPHKEHLYKLLVIGDLGVGKTSIIKRYVHQNF

SEQ ID NO: 5 - protein, 34 aa, Mus musculus; identical to SEQ ID NO: 4
  mouse RAB38 highly conserved N-terminal region

SEQ ID NO: 6 - protein, 47 aa, Homo sapiens
  human RAB3a N-terminal region
MASATDSRYGQKESSDQNFDYMFKILIIGNSSVGKTSFLFRYADDSF

SEQ ID NO: 7 - protein, 45 aa, Homo sapiens
  human RAB5 N-terminal region
MASRGATRPNGPNTGNKICQFKLVLLGESAVGKSSLVLRFVKGQF

SEQ ID NO: 8 - protein, 28 aa, Homo sapiens
  human N-RAS N-terminal region
MTEYKLVVVGAGGVGKSALTIQLIQNHF

SEQ ID NO: 9 - DNA, 1439 bp, Homo sapiens
  Rab38 cDNA
acatagagctccgggaaacgtcggtgcccagccccggctgtgcttcccagagcaagctccaggctccgcaagacccgcgggcctccaggatgcagacacctcacaaggagcacctgtacaagctgctggtgatcggcgacctgggtgtgggcaagaccagcattatcaagcgctatgtgcaccaaaacttctcctcgcactaccgggccaccattggtgtggacttcgcgctgaaggtgctccactgggacccagagacggtggtgcgcttgcagctctgggacattgctggtcaagaaagatttggaaacatgacaagagtttattaccgggaagctatgggggcatttattgtttttgatgtcaccagaccagccacatttgaagccgtggcaaagtggaaaaatgatttggactcaaagttaacgctccctaatggtaagccagtgtcagtggttctgttggccaacaaatgtgaccaagggaaggatgtgcttatgaacaatggactcaagatggaccagttctgcaaggagcatggcttcgtaggatggtttgaaacatcagccaaggaaaacataaacattgatgaagcctcaagatgcctggtcaagcacatacttgcaaatgagtgtgacctcctagagtctatagaaccggacattgtgaagccccatctcacatcgcccaaggttgtcagctgctctggctgtgccaaatcctagaaggctcctctgctggcatatgacagacagaacccgtggccctcatgaatcgtgcttcagtttttccttattaccattttgggtaagcgtcaggatagggaagcacatgtgacaagccaaagatacatgactgtatggttcctgtcaaagaggaacagcaaatgttctttatgtgttttcccaccccatcagcacagtgtttacaagcttttaaaatattagtctgtcacaatatgctgttttatcattgagcaaagccactcagggacacagacagccctaatatttgttcctttaaatcaacaaaggcttctggtcttcttgagaaggggaataacagagcaaggcagaggtcaagctaagtgtggggatttgtcttgccctggtgtgtctttgttcaggtatcaatttgttcccgggtggtctgataggtctattaaatagaaaccattcatggtagacctaagggttgkctgtgatgtttctcttcagagtcgtgtgcacaggcagcctgggcttttgttgtcacttgctgtgccctgaatgctggtttaactgaaaactgtatggaaagatctgctccctgtatgtgcctttctttcagcttcctctgactcaagctgcaggactcttctgtatgtggaagatatattatatatatttttcacaagtgaaaaataaaacattaaaaatgctgtttccctgtttctgata

SEQ ID NO: 10 - DNA, 291 bp, Homo sapiens
  Rab38 exon 1 and surrounding intron sequence
acatagagctccgggaaacgtcggtgcccagccccggctgtgcttcccagagcaagctccaggctccgcaagacccgcgggcctccaggatgcagacacctcacaaggagcacctgtacaagctgctggtgatcggcgacctgggtgtgggcaagaccagcattatcaagcgctatgtgcaccaaaacttctcctcgcactaccgggccaccattggtgtggacttcgcgctgaaggtgctccactgggacccagagacggtggtgcgcttgcagctctgggacattgctg

SEQ ID NO: 11 - DNA, 281 bp, Homo sapiens
  Rab38 exon 2
gtcaagaaagatttggaaacatgacaagagtttattaccgggaagctatgggggcatttattgtttttgatgtcaccagaccagccacatttgaagccgtggcaaagtggaaaaatgatttggactcaaagttaacgctccctaatggtaagccagtgtcagtggttctgttggccaacaaatgtgaccaagggaaggatgtgcttatgaacaatggactcaagatggaccagttctgcaaggagcatggcttcgtaggatggtttgaaacatcagccaag

SEQ ID NO: 12 - DNA, 868 bp, Homo sapiens
  Rab38 exon 3 and surrounding intron sequence
ggaaaacataaacattgatgaagcctcaagatgcctggtcaagcacatacttgcaaatgagtgtgacctcctagagtctatagaaccggacattgtgaagccccatctcacatcgcccaaggttgtcagctgctctggctgtgccaaatcctagaaggctcctctgctggcatatgacagacagaacccgtggccctcatgaatcgtgcttcagtttttccttattaccattttgggtaagcgtcaggatagggaagcacatgtgacaagccaaagatacatgactgtatggttcctgtcaaagaggaacagcaaatgttctttatgtgttttcccaccccatcagcacagtgtttacaagcttttaaaatattagtctgtcacaatatgctgttttatcattgagcaaagccactcagggacacagacagccctaatatttgttcctttaaatcaacaaaggcttctggtcttcttgagaaggggaataacagagcaaggcagaggtcaagctaagtgtggggatttgtcttgccctggtgtgtctttgttcaggtatcaatttgttcccgggtggtctgataggtctattaaatagaaaccattcatggtagacctaagggttgkctgtgatgtttctcttcagagtcgtgtgcacaggcagcctgggcttttgttgtcacttgctgtgccctgaatgctggtttaactgaaaactgtatggaaagatctgctccctgtatgtgcctttctttcagcttcctctgactcaagctgcaggactcttctgtatgtggaagatatattatatatatttttcacaagtgaaaaataaaacattaaaaatgctgtttccctgtttctgata

SEQ ID NO: 13 - DNA, 45 bp, Artificial Sequence
  Description of Artificial Sequencesynthetic TYRP15′T3F DNA
gcgcgaattaaccctcactaaagggtctgagcacccctgtcttct

SEQ ID NO: 14 - DNA, 45 bp, Artificial Sequence
  Description of Artificial Sequencesynthetic TYRP15′T7R DNA
gcgcgtaatacgactcactatagggcccagttgcaaaattccagt

SEQ ID NO: 15 - DNA, 47 bp, Artificial Sequence
  Description of Artificial Sequencesynthetic MLSN R T7 DNA
gcgggtaatacgactcactataggggccacaaacatgtcctacttac

SEQ ID NO: 16 - DNA, 44 bp, Artificial Sequence
  Description of Artificial Sequencesynthetic MLSN FT3 DNA
gcgcgaattaaccctcactaaagggaagcttccggactctctac

SEQ ID NO: 17 - DNA, 21 bp, Artificial Sequence
  Description of Artificial SequencePCR amplification primer Rab38 Ex1F
taggaaggaggattaaacccg

SEQ ID NO: 18 - DNA, 21 bp, Artificial Sequence
  Description of Artificial SequencePCR amplification primer Rab38 Ex 1R
gaactcctcatggctcactcc

SEQ ID NO: 19 - DNA, 26 bp, Artificial Sequence
  Description of Artificial SequencePCR amplification primer Rab38 Ex2F
ggatatgaagctccagtgtagtgtac

SEQ ID NO: 20 - DNA, 25 bp, Artificial Sequence
  Description of Artificial SequencePCR amplification primer Rab38 Ex2R
cactggacagaaacattattgtcac

SEQ ID NO: 21 - DNA, 26 bp, Artificial Sequence
  Description of Artificial SequencePCR amplification primer Rab38 Ex3F
aagttatcagccagtgagatactgtg

SEQ ID NO: 22 - DNA, 25 bp, Artificial Sequence
  Description of Artificial SequencePCR amplification primer Rab38 Ex3R
cacatgtggtatatctatcctgacg

SEQ ID NO: 23 - DNA, 21 bp, Artificial Sequence
  Description of Artificial Sequence amplification primer cht Ex1F
ggcctccaggatgcagacacc

SEQ ID NO: 24 - DNA, 21 bp, Artificial Sequence
  Description of Artificial Sequence amplification primer cht Ex1R
ccagcaatgtcccagagctgc

SEQ ID NO: 25 - DNA, 49 bp, Artificial Sequence
  Description of Artificial SequencePCR amplification att site linker primer AttB1-RRab
ggggacaagtttgtacaaaaaagcaggctccatgcagacacctcacaag

SEQ ID NO: 26 - DNA, 51 bp, Artificial Sequence
  Description of Artificial SequencePCR amplification att site linker primer AttB2-RRab-STP
ggggaccactttgtacaagaaagctgggttctaggatttggcacagccaga

SEQ ID NO: 27 - DNA, 15 bp, Artificial Sequence
  Description of Artificial Sequence oligonucleotide complementary to segment of RAB38 mRNA translation initiation codon
aacgttgaggggcat

SEQ ID NO: 28 - DNA, 1412 bp, Homo sapiens
  human RAB38 DNA sequence
ggctgcgcttccctggtcaggcacggcacgtctggccggccgccaggatgcaggccccgcacaaggagcacctgtacaagttgctggtgattggcgacctgggcgtggggaagaccagtatcatcaagcgctacgtgcaccagaacttctcctcgcactaccgggccacaatcggcgtggacttcgcgctcaaggtgctccactgggacccggagactgtggtgcgcctgcagctctgggatatcgcaggtcaagaaagatttggaaacatgacgagggtctattaccgagaagctatgggtgcatttattgtcttcgatgtcaccaggccagccacatttgaagcagtggcaaagtggaaaaatgatttggactccaagttaagtctccctaatggcaaaccggtttcagtggttttgttggccaacaaatgtgaccaggggaaggatgtgctcatgaacaatggcctcaagatggaccagttctgcaaggagcacggtttcgtaggatggtttgaaacatcagcaaaggaaaatataaacattgatgaagcctccagatgcctggtgaaacacatacttgcaaatgagtgtgacctaatggagtctattgagccggacgtcgtgaagccccatctcacatcaaccaaggttgccagctgctctggctgtgccaaatcctagtaggcacctttgctggtgtctggtaggaatgacctcattgttccacaaattgtgcctctatttttaccattttgggtaaacgtcaggatagatataccacatgtggcaagccaaagatctatgcctctgttttttcaatgagagagaaatagcaaatgttctttctatgctttcctcaccatcatcacagtgtttacaaacttttgaaaatatttagtctgttacaaacttctgtcatgtagctgaccaaaatcctgcagggccacagtcggcactgttatttgcttcttttaatcagcaaaggcctcaagtcttaaaataaaaggggagaagaacaaactagctgtcaagtcaaggactggctttcaccttgccctggtgtctttttccagatttcaatatattctctgatggcctgacaggcctattaagtagatgtgatattttcttccaagatgacctccattctcggcagacctaagagttgcctctgagttagctctttggaatcgtgaacacaggtgtgctatattgtccttgtcctaactgtcacttgccatggcctgaatgttggcttaactgaatattgtatgaaaagacatgcctccatatgtgcctttctgttagctctctttgactcaagctgtggggctcctctatacatgctatacatgtaatatatattatatatatttttgcaagtgaacaataaaacattaaaagataaaa